(19) United States
(12) Patent Application Publication
(10) Pub. No.: US 2024/0046071 A1
(43) Pub. Date: Feb. 8, 2024

(54) FEATURES EXTRACTION NETWORK FOR ESTIMATING NEURAL ACTIVITY FROM ELECTRICAL RECORDINGS

(71) Applicant: California Institute of Technology, Pasadena, CA (US)

(72) Inventors: Tyson Aflalo, Pasadena, CA (US); Benyamin A Haghi, Pasadena, CA (US); Richard A Andersen, Pasadena, CA (US); Azita Emami, Pasadena, CA (US)

(73) Assignee: California Institute of Technology, Pasadena, CA (US)

(21) Appl. No.: 18/230,448

(22) Filed: Aug. 4, 2023

Related U.S. Application Data
(60) Provisional application No. 63/395,231, filed on Aug. 4, 2022.

(51) Int. Cl. G06N 3/045; G16H 40/67; G16H 50/30; G06N 3/084; G06F 3/01

(52) U.S. Cl. CPC G06N 3/045; G16H 40/67; G16H 50/30; G06N 3/084; G06F 3/015

(57) ABSTRACT

An apparatus and method for a feature extraction network based brain machine interface is disclosed. A set of neural sensors sense neural signals from the brain. A feature extraction module is coupled to the set of neural sensors to extract a set of features from the sensed neural signals. Each feature is extracted via a feature engineering module having a convolutional filter and an activation function. The feature engineering modules are each trained to extract the corresponding feature. A decoder is coupled to the feature extraction module. The decoder is trained to determine a kinematics output from a pattern of the plurality of features. An output interface provides control signals based on the kinematics output from the decoder.

### 1. PRIORITY CLAIM

This disclosure claims priority to and the benefit of U.S. Provisional Application No. 63/395,231, filed Aug. 4, 2022. The contents of that application in their entirety are hereby incorporated by reference.

### 2. TECHNICAL FIELD

The present disclosure relates to feature extraction from brain signals, and specifically to a feature extraction network that is trained to provide features from electrical signals received from neural sensors.

### 3. BACKGROUND

The following description includes information that may be useful in understanding the present invention. It is not an admission that any of the information provided herein is prior art or relevant to the presently claimed invention, or that any publication specifically or implicitly referenced is prior art.

Brain machine-interface (BMI) technologies communicate directly with the brain and can improve the quality of life of millions of patients with brain circuit disorders. Motor BMIs are among the most powerful examples of BMI technology. Ongoing clinical trials implant microelectrode arrays into motor regions of tetraplegic participants. Movement intentions are decoded from recorded neural signals into command signals to control a computer cursor or a robotic limb. Clinical neural prosthetic systems enable paralyzed human participants to control external devices by: (a) transforming brain signals recorded from implanted electrode arrays into neural features; and (b) decoding neural features to predict the intent of the participant. However, these systems fail to deliver the precision, speed, degrees-of-freedom, and robustness of control enjoyed by motor-intact individuals. To enhance the overall performance of the BMI systems and to extend the lifetime of the implants, newer approaches for recovering functional information of the brain are necessary.

Part of the difficulty of improving BMI control is the unconstrained nature of the design problem of the interface system. The interface system design can be fundamentally modeled as a data science problem: the mapping from brain activity to motor commands must be learned from data and must find adequate solutions to the unique challenges of neural interfaces. These problems include limited and costly training data, low signal-to-noise ratio (SNR) predictive features, complex temporal dynamics, non-linear tuning curves, neural instabilities, and the fact that solutions must be optimized for usability, not offline prediction. These properties have made end-to-end solutions (e.g., mapping 30 KHz sampled array recordings to labeled intention data) intractable. Therefore, most BMI systems separate the decoding problem into two distinct phases: (1) transforming electrical signals recorded from implanted electrode arrays into neural features; and (2) learning parameters that map neural features to control signals. Current studies usually compare the decoders across a limited set of feature extraction techniques, such as neural threshold crossings (TCs) or wavelets (WTs). However, most of these feature extraction techniques, including TCs and WTs, are suboptimal since they use simple heuristics or were developed in other domains and simply applied to the neural signals. Therefore, these methods may perform sub-optimally compared to the data-driven methods that may better account for the specific biophysical processes giving rise to the dynamics of interest in the raw electrical recordings. The process of learning an optimal mapping from raw electrical recordings to neural features has not been explored.

Improving estimates of neural activity based on measured electrical signals has been largely unexplored. The need for new approaches is critical in order to more accurately translate neural activity to reflect the intent of the user. Accurately recovering functional information from implanted electrodes over time may extend the lifetime of electrode implants to reduce the need for subsequent brain surgeries.

Several current methods for recovering function information include: 1) counting the number of neural spiking events per unit time as detected by when a neural waveform cross a threshold on filtered broadband neural recordings (termed threshold crossings); 2) counting the number of neural spiking events per unit time after template matching waveforms; 3) counting the number of neural spiking events per unit time after sorting crossings based on waveform shape; 4) computing the total power in the filtered broadband signal over a fixed window of time; and 5) computing the power of a frequency decomposed signal as computed using wavelets, windowed Fourier transforms, or multi-taper Fourier transforms. Unfortunately, these existing techniques all suffer from potential future inaccuracy as the implant electrodes age.

In addition, neural decoding relies on having accurate estimates of neural activity. Implantable electrode-based BMIs promise to restore autonomy to paralyzed individuals if they are sufficiently robust and long-lasting to overcome the inherent risks associated with brain surgery. Unfortunately, the breakdown of materials in the hostile environment of the body and inherent stochasticity of the quality of information available at individual electrodes provide a significant hurdle for the safety and efficacy of implantable solutions. Currently, using existing approaches, the ability to recover functional brain signals from electrical recordings degrades over time, becoming unusable after 3-7 years post-implantation of the electrode arrays. Innovations in material sciences, minimally invasive delivery, and novel design provide one path to overcome these limitations, but they may take many years to receive FDA approval and may not improve baseline decoding quality.

Fluctuations in electrical activity recorded at an electrode come from a diversity of sources. Typically, a neural decoding pipeline starts with extracting a particular neural feature of interest, which has historically been the number of neural spikes per unit of time. However, recent work has shown that alternative ways of processing broadband electrical recordings (e.g., wavelet decompositions or power) can improve the information content of extracted features.

Thus there is a need for a system that provides robust feature extraction from neural signals for accurate prediction of neural responses over time. There is another need for a feature extraction system that may be used with existing implants and decoders. There is also a need for a feature extraction network that may be adapted to different patients and different applications.

### 4. SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

One example is a brain interface system including a set of neural signal sensors sensing neural signals from a brain. A feature extraction module includes a plurality of feature engineering modules each coupled to the set of neural signal sensors. The feature engineering modules are trained to extract a plurality of features from the sensed neural signals. A decoder is coupled to the feature extraction module. The decoder determines a brain state output from a pattern of the plurality of features.

In another implementation of the disclosed example system, the brain state output is a kinematics control. The system includes an output interface providing control signals based on the kinematics output from the decoder. In another implementation, the output interface is a display and the control signals manipulate a cursor on a display. In another implementation, the example system includes a mechanical actuator coupled to the output interface. The control signals manipulate the mechanical actuator. In another implementation, the set of neural signal sensors is one of a set of implantable electrodes or wearable electrodes. In another implementation, the brain state output is an indication of a brain disorder. In another implementation, each of the feature engineering modules include an upper convolutional filter coupled to the neural signal sensors and an activation function to output a feature from the neural signal sensors. In another implementation, each of the feature engineering modules include a lower convolutional filter coupled to the neural signal sensors. The lower convolutional filter outputs an abstract signal to a subsequent feature engineering module. The lower convolutional filter of a last feature engineering module outputs a final feature. In another implementation, each of the plurality of feature engineering modules use identical parameters for all neural signal sensors used in a training data set for training the feature engineering modules. In another implementation, each of the plurality of feature engineering modules include an adaptive average pooling layer coupled to the activation function to summarize a pattern of features into a single feature. In another implementation, the example system includes a partial least squares (PLS) regression module coupled to the output of the feature extraction module. The PLS regression module reduces the plurality of features to a subset of features. In another implementation, the feature extraction module includes a fully-connected layer of nodes to reduce the plurality of features to a subset of features. In another implementation, the training of the feature engineering modules includes adjusting the convolutional filters from back propagation of error between the brain state output of the decoder from a training data set and a desired brain state output. In another implementation, the decoder is one of a linear decoder, a Support Vector Regression (SVR) decoder, a Long-Short Term Recurrent Neural Network (LSTM) decoder, a Recalibrated Feedback Intention-Trained Kalman filter (ReFIT-KF) decoder, or a Preferential Subspace Identification (PSID) decoder. In another implementation, a batch normalization is applied to the inputs of a training data set for training the feature engineering modules.

Another example is a method of deriving features from a neural signal for determining brain state signals from a human subject. A plurality of neural signals is received from the human subject via a plurality of neural signal sensors. Features from the plurality of neural signals are determined from a feature extraction network having a plurality of feature engineering modules, each trained to extract a feature from the neural signals.

In another implementation of the disclosed example method, the features are decoded via a trained decoder to output brain state signals to an output interface. In another implementation, the brain state output is a kinematics control, and the output interface provides control signals based on the kinematics output from the decoder. In another implementation, the output interface is a display and the control signals manipulate a cursor on a display. In another implementation, the control signals manipulate a mechanical actuator coupled to the output interface. In another implementation, the brain state output is an indication of a brain disorder. In another implementation, the plurality of neural signal sensors is one of a set of implantable electrodes or wearable electrodes. In another implementation, each of the feature engineering modules include an upper convolutional filter coupled to the neural signal sensors and an activation function to output a feature from the neural signal sensors. In another implementation, each of the feature engineering modules include a lower convolutional filter coupled to the neural signal sensors. The lower convolutional filter outputs an abstract signal to a subsequent feature engineering module.

The lower convolutional filter of a last feature engineering module outputs a final feature. In another implementation, each of the feature engineering modules use identical parameters for all neural signal sensors used in a training set for training the feature engineering modules. In another implementation, each of the plurality of feature engineering modules include an adaptive average pooling layer coupled to the activation function to summarize a pattern of features into a single feature. In another implementation, the example method reduces the plurality of features to a subset of features using a partial least squares (PLS) regression module coupled to the output of the feature extraction module. In another implementation, the example method reduces the plurality of features to a subset of features using a fully-connected layer of nodes of the feature extraction network. In another implementation, the training of the feature engineering modules includes adjusting the convolutional filters from back propagation of error between the brain state output of the decoder from a training data set and a desired brain state output. In another implementation, the decoder is one of a linear decoder, a Support Vector Regression (SVR) decoder, a Long-Short Term Recurrent Neural Network (LSTM) decoder, a Recalibrated Feedback Intention-Trained Kalman filter (ReFIT-KF) decoder, or a Preferential Subspace Identification (PSID) decoder. In another implementation, a batch normalization is applied to the inputs of a training data set for training the feature engineering modules.

Another example is a non-transitory computer-readable medium having machine-readable instructions stored thereon, which when executed by a processor, cause the processor to receive a plurality of neural signals from the human subject via a plurality of neural sensors. The instructions cause the processor to determine features from the plurality of neural signals from a feature extraction network having a plurality of feature engineering modules. Each of the feature engineering modules is trained to extract a feature from the neural signal. The instructions cause the processor to decode the features via a trained decoder to output brain state signals to an output device.

Another example is a method for training a feature extraction network having a plurality of feature engineering modules to output features from neural inputs. A training data set of neural signals from a brain of a subject and desired features corresponding to the neural signals is assembled. A decoder is trained to output a desired brain state from the desired features. The feature extraction network is trained to extract the desired features from a neural signal with the training data set. Each feature engineering module is trained to extract a feature from the neural signal.

In another implementation of the disclosed example method, the training data set is derived from signals from a plurality of electrodes in contact with the brain of the subject. In another implementation, the training data set is derived from a subset of electrodes having the highest performance on a validation data set. In another implementation, the features output by the feature engineering modules is a set of features determined by wavelet decomposition of the neural signals. In another implementation, the desired features in the training data set relate to one of kinematics control or an indicator of a brain disorder. In another implementation, the electrodes are in one of a brain implant or a wearable. In another implementation, each of the feature engineering modules include an upper convolutional filter coupled to the neural inputs and an activation function to output a feature from the neural inputs. In another implementation, each of the feature engineering modules include a lower convolutional filter coupled to the neural signal sensors. The lower convolutional filter outputs an abstract signal to a subsequent feature engineering module. The lower convolutional filter of a last feature engineering module outputs a final feature. In another implementation, the training includes updating the upper convolutional filter of each feature engineering module through back propagating error between a base line brain state output and the output of the decoder. In another implementation, each of the plurality of feature engineering modules use identical parameters for all neural signal sensors used in the training data set.

Another disclosed example is a system for training a feature extraction network having a plurality of feature engineering modules to output features from neural inputs. The system includes a storage device storing a training data set of neural signals from a brain of a subject and desired features corresponding to the neural signals. A processor is coupled to the storage device. The processor is operable to input a set of neural signals to the plurality of feature engineering modules. The processor is operable to read a set of features output by plurality of feature engineering modules. The processor is operable to decode the set of features to a brain state via a trained decoder. The processor is operable to compare the decoded brain state with a desired brain state from the training data set to determine an error. The processor is operable to iterate a parameter of the feature engineering modules based on the error. The processor is operable repeat the reading and comparing until each of the feature engineering modules are trained to extract a feature from the neural signal.

In another implementation of the disclosed example system, the training data set is derived from signals from a plurality of electrodes in contact with the brain of the subject. In another implementation, the training data set is derived from a subset of electrodes having the highest performance on a validation data set. In another implementation, the training data set is derived from signals from a plurality of electrodes in contact with the brain of the subject. In another implementation, the features output by the feature engineering modules is a set of features determined by wavelet decomposition of the neural signals. In another implementation, the desired features in the training data set relate to one of kinematics control or an indicator of a brain disorder. In another implementation, the electrodes are in one of a brain implant or a wearable. In another implementation, each of the feature engineering modules include an upper convolutional filter coupled to the neural inputs and an activation function to output a feature from the neural inputs. In another implementation, each of the feature engineering modules include a lower convolutional filter coupled to the neural signal sensors. The lower convolutional filter outputs an abstract signal to a subsequent feature engineering module. The lower convolutional filter of a last feature engineering module outputs a final feature. In another implementation, iterating the parameter includes updating the upper convolutional filter of each feature engineering module through back propagating error between the desired brain state and the brain state output of the decoder. In another implementation, each of the plurality of feature engineering modules use identical parameters for all neural signal sensors used in the training data set.

Another disclosed example is a non-transitory computer-readable medium having machine-readable instructions stored thereon. The instructions, which when executed by a processor, cause the processor to assemble a training data set of neural signals from a brain of a subject and desired features corresponding to the neural signals. The instructions cause the processor to train a decoder to output a desired brain state from the desired features. The instructions cause the processor to train the feature extraction network to extract the desired features from a neural signal with the training data set. Each feature engineering module is trained to extract a feature from the neural signal.

### 5. BRIEF DESCRIPTION OF DRAWINGS

In order to describe the manner in which the above-recited disclosure and its advantages and features can be obtained, a more particular description of the principles described above will be rendered by reference to specific examples illustrated in the appended drawings. These drawings depict only example aspects of the disclosure, and are therefore not to be considered as limiting of its scope. These principles are described and explained with additional specificity and detail through the use of the following drawings:

FIG. 1 shows an example brain machine interface system using an implant, a feature extraction module, and decoder control system for moving a cursor on a display;

FIG. 2 is an example block diagram of the example feature extraction module and decoder module in FIG. 1;

FIG. 3A shows an example feature extraction network in FIG. 2;

FIG. 3B shows an example feature engineering network module in FIG. 3B;

FIG. 3C shows an example training system with example data, according to one or more embodiments of the present disclosure;

FIG. 4A shows maps of cursor positions from tests conducted on a brain machine interface using an example feature extraction network and a brain machine interface using known threshold crossings control;

FIG. 4B is a set of graphs of resulting measurements for tests conducted on a brain machine interface using an example feature extraction network and a brain machine interface using known threshold crossings control;

FIG. 4C is a set of graphs showing distance to target and speed measurements for tests conducted on a brain machine interface using an example feature extraction network and a brain machine interface using known threshold crossings control;

FIG. 4D is a set of graphs showing success rate and bit rate for tests conducted on a brain machine interface using an example feature extraction network and a brain machine interface using known threshold crossings control;

FIG. 5A shows graphs that show coefficient of determination data for different tests for a first subject using the example feature extraction network against other types of feature extraction methods;

FIG. 5B shows graphs that show coefficient of determination data for different tests for a second subject using the example feature extraction network against other types of feature extraction methods;

FIG. 5C shows velocity graphs for an experimental subject using the example feature extraction network against other types of feature extraction methods;

FIG. 6A shows graphs that plot cross-validated coefficient of determination using the example feature extraction network against other types of feature extraction methods for a first participant;

FIG. 6B shows graphs that plot cross-validated coefficient of determination using the example feature extraction network against other types of feature extraction methods for a second participant;

FIG. 7A shows graphs of the coefficient of determination at different cutoff frequencies;

FIG. 7B shows graphs of the performance of the example feature extraction network with and without a partial least-squares regressor;

FIG. 8A shows a series of graphs showing comparisons of cross validated coefficient of determinations from different feature extraction techniques;

FIG. 8B shows a series of graphs showing magnitude and phase differences for different electrodes;

FIG. 9A shows a series of graphs showing coefficient of determinations for different decoders used in conjunction with the example feature extraction network and other known feature extraction methods for a first participant;

FIG. 9B shows a series of graphs showing coefficient of determinations for different decoders used in conjunction with the example feature extraction network and other known feature extraction methods for a second participant;

FIG. 10A shows a series of graphs showing different strides in relation to cross validated coefficient of determination for the example feature extraction network;

FIG. 10B shows a series of graphs showing different kernels in relation to cross validated coefficient of determination for the example feature extraction network;

FIG. 10C shows graphs relating to computational cost and impact on prediction for different hyperparameters for the example feature extraction network;

FIG. 11A shows a series of graphs of the average values for features in a first electrode extracted by the example feature extraction network in comparison to other feature methods for different sessions;

FIG. 11B shows a series of graphs of the actual values for features in a first electrode extracted by the example feature extraction network in comparison to other feature methods;

FIG. 12A shows a series of graphs plotting gain of example convolutional filters in comparison to known feature methods;

FIG. 12B shows a series of graphs plotting the broadband neural data amplitude for samples for the example feature extraction network and a known feature method;

FIG. 13 shows a series of charts of the decode performance of a participant using the example feature extraction network over different time periods and for different brain regions;

FIG. 14A is a schematic representation of a finger-grid task used for testing the example feature extraction network;

FIG. 14B shows a series of graphs showing the results of the finger-grid tasks from using the example feature extraction network compared with a known feature method;

FIG. 15 shows a series of graphs testing the effect of smoothing on the outputs of the example feature extraction network;

FIG. 16A shows a graph of the amount of training sessions to properly train the example feature extraction network;

FIG. 16B shows tables showing training and testing for electrodes in the array for the example feature extraction network;

FIG. 16C shows graphs showing the performance of a linear decoder operating on partial least square analysis;

FIG. 17 shows graphs comparing the application of a PLSR to the example feature extraction network; and

FIG. 18 is an example control system for brain control of a mechanical actuator incorporating the example feature extraction network.

### 6. DETAILED DESCRIPTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials specifically described.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

The present disclosure relates to a method and system to extract features for accurate estimation of neural activity such as brain state from recorded electrical neural signals. The method works by learning an optimized mapping between electrical signals and neural features from subjects. Neural features define the activity of local populations of neurons but do not yet specify the functional information the neurons carry. This method is parameterized using an architecture that jointly optimizes the feature extraction and feature decoding stages of the neural decoding process. This architecture ensures that the neural features extracted by the algorithm maximize the amount of functional information carried by the neural features. Further, the feature extraction algorithm is constrained to use the same parameters for all neural sensors such as electrodes used in the training set, thus finding a solution that is able to generalize to new recordings, even if these recordings should occur using different electrodes recorded from different individuals.

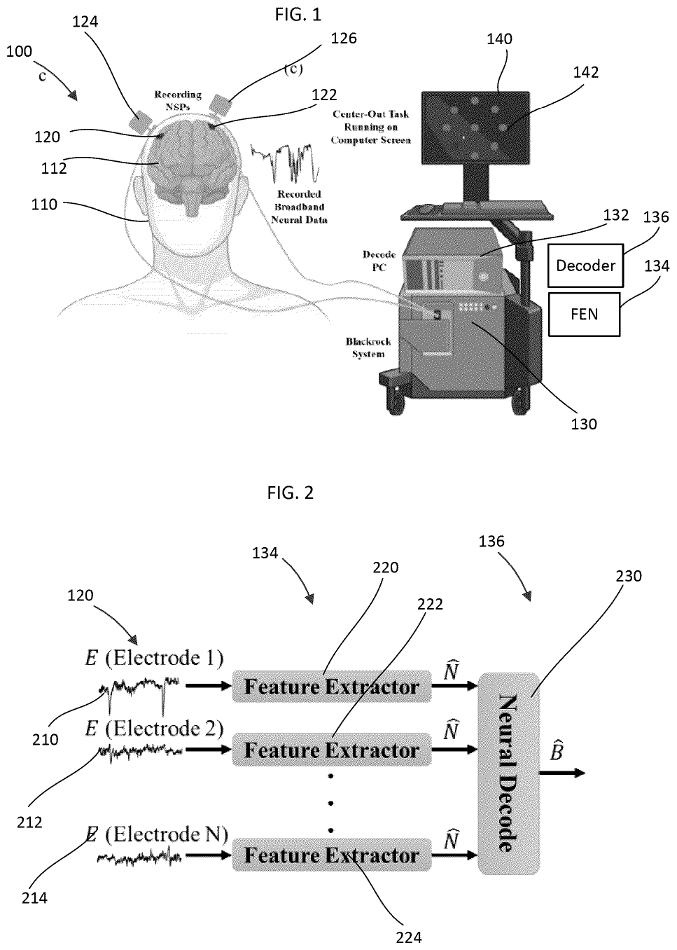

FIG. 1 is an example system 100 that allows a user 110 to manipulate a cursor via neural signals from the brain 112. The neural signals are input into neural sensors and the system 100 allows for the output of a brain state. The system 100 includes neural sensors such as electrodes in two neural implants 120 and 122. Each of the neural implants 120 and 122 communicate signals to neural signal processor (NSP) modules 124 and 126 respectively. The neural implants 120 and 122, and NSP modules 124 and 126 may be Utah microelectrode arrays in the NeuroPort product available from Blackrock Microsystems. The outputs from the NSP modules 124 and 126 are sent to a signal processing system 130. In this example, the signal processing system 130 is a Cereplex Direct unit available from Blackrock Neurotech. A decoder computer 132 executes a trained feature extraction network based feature extraction module 134 and a neural decoding module 136. After the pre-processing, the decode computer 132 extracts the appropriate neural features and decodes the neural features to the computer cursor movements. An output interface such as a display 140 is coupled to the decode computer 132. The user 110 may manipulate a cursor on the display 140 and select certain areas of a graphic 142 via the system 100.

In this example, each of the implants 120 and 122 has a series of electrodes that detect neural signals from the brain 112. In this example, the implant 120 is implanted in the Motor Cortex (M1) region and the implant 122 is implanted in the Posterior Parietal Cortex (PPC) of the brain 112. In this example, each of the implants 120 and 122 has 96 separate electrodes that provide neural signals. In this example, each of the electrodes records the broadband data that consists of various neural activities (e.g., somata, dendrites, axons, etc). Neurons close to the electrode will generate stronger single-unit activities compared to the neurons far from the recording electrode, which record multi-unit neural activities (MUA). The electrode records noise as the distance of the neurons increases. The NSP modules 124 and 126 receive the broadband signals from the electrodes of the respective implants 120 and 122.

FIG. 2 is a block diagram of the architecture of the control system 100 in FIG. 1. The architecture in FIG. 2 enables training of the example feature extraction network for feature generation. A neural decoder may also be trained by the control system 100. The implant 120 includes a series of electrodes 210, 212, and 214 that may detect a number of neural signals, N. The control module 134 in FIG. 1 includes a series of fixed feature mapping modules 220, 222, and 224. Each of the electrodes 210, 212, and 214 is coupled to one of the respective fixed feature mapping modules 220, 222, and 224. Each of the fixed feature mapping modules 220, 222, and 224 estimate neural activity \hat{N} from recorded electrical activity. The example system has fixed parameters for all the electrodes, tasks, sessions, and participants. The outputs of the fixed feature mapping modules 220, 222, and 224 are coupled to a neural decoder module 230. The neural decoder module 230 estimates the behavior \hat{B} from estimates of the neural activity \hat{N}. The output of the decoder module 230 are control signals for an output device such as a cursor or a mechanical actuator. In this example, the neural decoder module 230 is learned for each training session of the fixed feature mapping modules 220, 222, and 224.

In this example, the feature matching modules 220, 222, and 224 produces a mapping from electrical fluctuations, E, received from the electrodes 210, 212, and 214 to estimate neural activity \hat{N}. However, there is no direct knowledge of actual neural activity, \hat{N}, so the feature matching modules 220, 222, and 224 find the neural activity, \hat{N} that optimizes estimates of the behavioral state.

The parameters mapping electrical fluctuations, E, to neural activity, \hat{N} are fixed across all electrodes, participants, and recording sessions. The mapping between \hat{N} and behavior, \hat{B} is variable between datasets given known nonstationarities, differences between subjects, etc. By constraining the complexity of the mapping between \hat{N} and \hat{B}, by only allowing a linear mapping for instance, the nonlinear mapping from E to \hat{N} is encouraged to be maximally descriptive. To learn this mapping, the example process uses a compact feature extraction network that learns an optimized mapping between electrical signals and neural features.

Parameters mapping E to \hat{N} across all electrodes and recording sessions are fixed, while allowing the mapping between the estimate of the neural activity \hat{N} to behavior \hat{B} (such as cursor velocity) to be electrode and session dependent. This approach assumes that the same transfer function can be applied to all electrodes and is independent of the relationship between the neural state and the behavior. Sharing weights across electrodes reduces the number of parameters, improves interpretability, and encourages solutions that generalize to new electrodes with distinct tuning properties.

One example feature extraction network termed FENet is designed as a multi-layer 1D convolutional architecture for the feature extraction module. Similar to the system in FIGS. 1-2, the feature extraction network FENet maps from E to \hat{N}, while using a linear mapping that decodes the estimates of neural activity, \hat{N}, to the behavior, \hat{B}. The use of a linear mapping is designed to encourage maximum learning within the example feature extraction network.

In one example, a two-stage optimization problem is created that transforms broadband signals into movement kinematics within a brain-machine interface cursor control paradigm. In the first stage, broadband activity is transformed into neural features using the example convolutional network as a 1-D convolutional neural network. In the second stage, an analytic linear mapping is trained to predict movement kinematics from the resulting neural features. The two-stage joint optimization enforces that the feature extraction process generates informative features while being independent of the relationship between neural activity and cursor kinematics. Since each electrode records a relatively independent one-dimensional temporal signal, one-dimensional convolutional filters are used in the feature extractor architecture in the system 100 in FIG. 1 to take in single-electrode broadband samples and output M features (i.e., the instantaneous states of the various information sources on the electrode).

The example feature extraction network FENet is unique since it is parameterized using a novel architecture that jointly optimizes the feature extraction and feature decoding stages of the neural decoding process, while constraining the feature extraction algorithm to use the same parameters for all the electrodes used in the training set. Moreover, the example FENet receives a single neural channel of broadband data as its input and extracts the most informative features of a signal automatically. This process can be repeated for all recording channels to estimate the current state of a neural population. As a nonlinear feature extractor, the example FENet consists of a set of convolutional filters, nonlinear activation functions, and pooling layers.

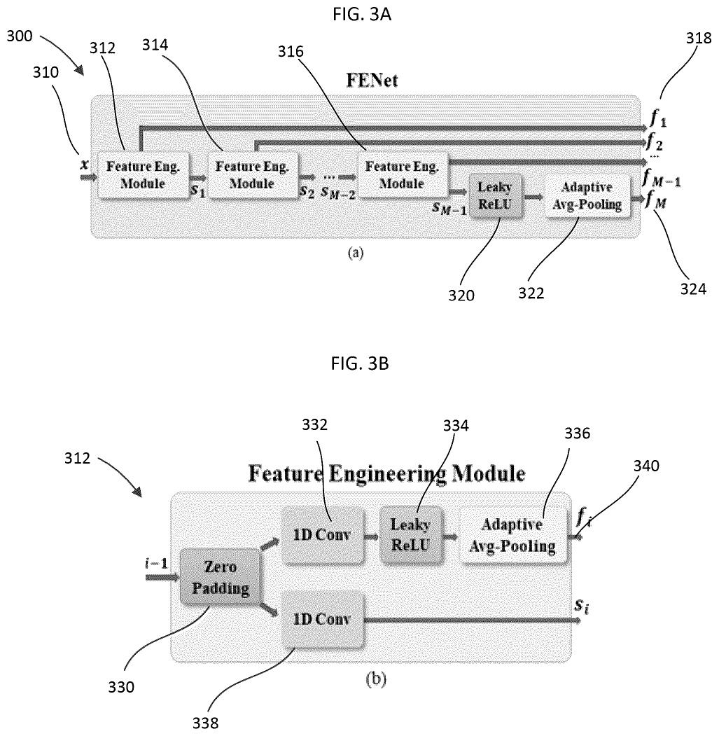

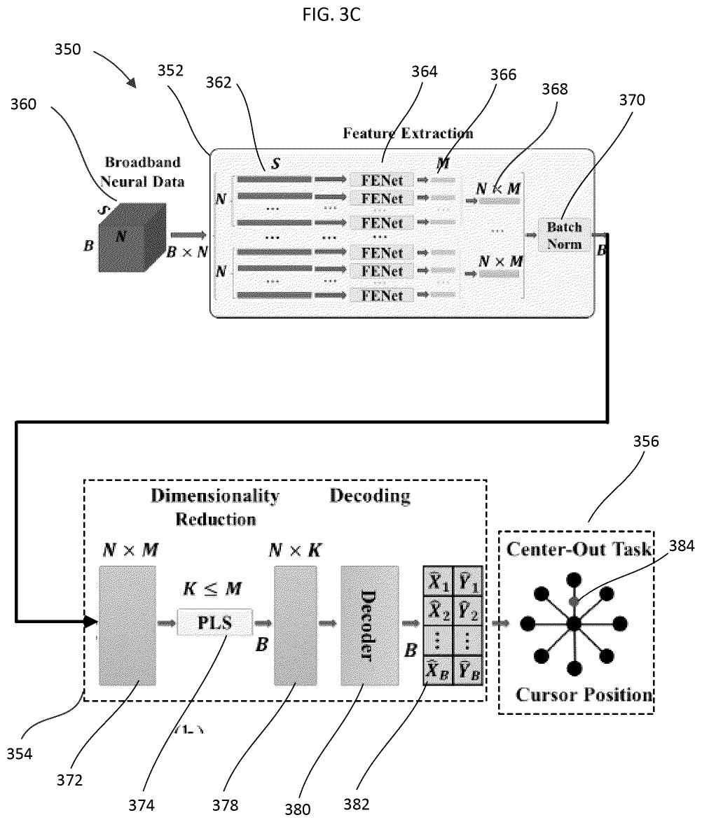

FIG. 3A shows an implementation of a feature extraction network 300 that may be in one of the feature extraction modules such as the feature extraction module 212 in FIG. 2. An input signal 310 of an electrode, x, is fed into a series of feature engineering modules 312, 314, and 316. Each of the feature engineering modules 312, 314, and 316 extract a certain feature from the input signal 310 via a convolutional filter and an activation function. In this example, eight features may be extracted and thus there are seven feature engineering modules. Each of the extracted features is output in a series of feature outputs 318. The input signal to each of the feature engineering modules is also passed on to the next feature engineering module through another convolutional filter. The last input signal from the last feature engineering module 316 is fed into a leaky rectified linear unit (ReLU) 320. The output of the ReLU 320 is fed in an adaptive average features module 322 that outputs a feature output 324 constituting the final feature (eighth feature in this example).

FIG. 3B shows a block diagram of one the feature engineering modules such as the feature engineering module 312 in FIG. 3A. The example feature engineering module 312 includes a padding module 330, an upper one-dimensional convolutional filter 332, a leaky rectified linear unit (ReLU) activation function 334, and adaptive average pooling unit 336. A feature output 340 is determined by the convolutional filter 332. A second lower convolutional filter 338 provides an abstract output signal to the next feature engineering module such as the feature engineering module 314 in FIG. 3A. The architecture of FENet in the BMI system 350 in FIG. 3C. Let ∈ [symbol] denote the input of the FENet with size 1×S, where S is the number of input data samples. The input is passed into M−1 back-to-back feature engineering modules such as the feature engineering modules 312, 314, and 316 in FIG. 3A.

In each feature engineering module, the input data of the i^{th} feature engineering module, sᵢ₋₁, is padded with zeros via the zero padding module 330, and the zero-padded data is passed through the two separate temporal 1-D convolutional filters 332 and 338. The output of the upper convolutional filter 332 is downsampled by stride 2 and is passed through the leaky ReLU nonlinear activation function 334. The leaky ReLU activation function 334 is designed to find the absolute value of its input with the parameter α=−1 in the negative side. Then, the output of the current filter is passed through the adaptive average pooling layer to summarize extracted temporal patterns into a single feature, fᵢ. The output of the lower convolutional filter 338 is passed to the next feature engineering module. This process is repeated to find the output feature vector. The output of the lower filter of the last feature engineering module is passed to the leaky ReLU activation function 320 and the adaptive average pooling layer 322 to append this single extracted feature to the feature vector as well. Therefore, the upper convolutional filter in each feature engineering module generates one of the FENet extracted features and the lower convolutional filter of each module extracts more abstract features from its input to be used as the input of the next feature engineering module. Finally, batch normalization is used as a regularization technique, which standardizes the output of the last layer of FENet to zero mean and unit variance for the training examples equal to the batch size. Batch normalization helps the employed optimization algorithm by keeping inputs closer to the normal distribution during the training process. The example feature extraction network is unique since it is parameterized using a novel architecture that jointly optimizes the feature extraction and feature decoding stages of the neural decoding process, while constraining the feature extraction algorithm to use the same parameters for all the electrodes used in the training set. The constraint of sharing parameters across electrodes will keep the number of learnable parameters small in the example architecture. Moreover, the feature engineering modules of the example feature extraction network are trained to receive a single neural electrode of broadband data as an input and the modules extract the most informative features of the signal automatically. The feature engineering modules are trained to output the features and the output features of a training data set is used as a baseline. In this example, an initial set of wavelet decomposition features are used for selection of the initial feature engineering modules. The resulting features are used to train the decoder in a particular session to output a desired brain state output from the features. Then, the decoder generates the desired brain state outputs for the application such as movement kinematics. Other applications such as brain disease detection may have different desired outputs. The error is calculated between the baseline of intended desired outputs and the decoded outputs from the features. In this example, regression error, which is mean square error (MSE) is calculated but other methods such as classification of regression error may be used. The convolutional filters are then updated using backpropagation through the feature extraction network. In this example, the error is back propagated through a gradient descent process such as Adams to obtain new values for the convolutional filters of the engineering modules and the set of neural signal inputs is fed into the feature engineering modules to output a set of features. The validation error is evaluated to select a model with the smallest validation error as the trained model using early stopping. Early stopping looks at the validation error and if the validation error starts to increase, training is stopped after a set number of steps. The model with the smallest validation error is then selected. This process can be repeated for all recording electrodes to estimate the current state of a neural population independent from the decoder.

FIG. 3C shows an architecture of a brain machine interface (BMI) system 350 that includes a feature extraction module 352, a decoder 354, and an output interface, which is a display 356. In this example, the display 356 includes a cursor that may be moved on a graphic on the display 356 via a user of the BMI system 350. The feature extraction module 352 includes the design of the feature extraction network 300 in FIG. 3A and the feature engineering modules such as the feature engineering module 312 in FIG. 3B with zero padding, 1-D convolutional filters, a leaky ReLU activation function, and adaptive average pooling. The architecture of the BMI system 350 accepts a set of input broadband neural data 360 that is sampled from the electrodes on an implant that is placed in proximity to neurons of the brain. Alternatively, a fully connected layer may be added as the last layer of the feature extraction module for dimensionality reduction. In the example feature extraction network FENet, there are 7 convolutional layers with convolutional filters with a filter size of 40. The zero-padding length for each feature engineering module is 39. The stride of each of the convolution filters was 2 with one input channel and one output channel. The 7 convolutional layers, each with 2 filters, with a filter size 40 result in 7*2*40=560 trainable parameters for the example feature extraction network of the FENet. These numbers can change based on the application and desired features to be extracted from the signal. As explained herein, a Bayesian optimization nay be performed for a parameter sweep to provide the best settings of the network for different applications.

The input 360 of the system is the broadband neural data with the dimension of B×N×S, where B is the batch size, N is the number of input neural electrodes, and S is the number of samples of the broadband neural data in a specific time interval. Each set of sample signals 362 is sent to a set of feature extraction networks 364. Each of the feature extraction networks in the set of feature extraction networks 364 may have a similar architecture as that in FIG. 3B. To update the network parameters during training, one training session was selected and a batch of the associated broadband activities was passed to the example feature extraction network to extract neural features. According to experiments, the best performance was achieved when the batch size was set to be equal to the length of a session. Moreover, one training session was used for each update cycle as it is the only way that simultaneously acquired neural recordings can be associated with corresponding cursor kinematics. The same FENet parameters are applied to all the neural electrodes. The outputs of the feature extraction networks 364 include a set of M generated neural features 366 for each electrode. The set of neural features 366 are grouped into the N outputs 368. A batch normalizer 370 outputs the B batches.

A feature matrix 372 is assembled from the output of the batch normalizer 370 with the dimension of B×(N×M). This feature generation process is the first stage of the two-stage optimization process. To reduce the dimension of the output per channel to avoid overfitting of the consequent decoder, an electrode specific partial least-squares regressor (PLSR) 374 is applied to the features generated by the set of feature extraction networks of each neural electrode to reduce the M features to K features, in which K≤M. For example, eight features could be reduced to two features. A reduced set of N×K×B features is passed to a decoder 380. The decoder 380 is an analytical linear decoder, which learns to map the extracted neural features to the movement kinematics (382). In this example, the neural features are mapped to movements of a computer cursor 384 on the display 140.

The example FENet feature extraction architecture was validated by predicting the kinematics of a computer cursor using neural data recorded from electrode arrays implanted in the human cortex such as those in the system 100 in FIG. 1. Such decoding was performed both on “offline” neural data acquired on previously recorded data and “online” as the basis of creating neural features that are subsequently decoded into control signals that allow the human patient to control a computer cursor under visual feedback. The performance of neural features extracted by the example FENet were compared against two current gold standards: 1) the rate of neural spike events computed by counting threshold crossings of the broadband neural signal; and 2) the wavelet decomposition of the broadband neural data.

An FDA- and IRB-approved brain machine interface study was conducted with a first 54-year-old (referred to as JJ) and a second 32-year-old (referred to as EGS) tetraplegic (C5-C6) male human research participants for trajectory tasks. The participant JJ had Utah microelectrode arrays (NeuroPort, Blackrock Microsystems, Salt Lake City, UT, USA) implanted in the hand-knob of the motor cortex and superior parietal lobule of the posterior parietal cortex (PPC). The participant EGS had Utah electrode arrays implanted near the medial bank of Anterior Intraparietal Sulcus (AIP) and in Broadman's Area 5 (BA5). Open-loop data over 54 sessions were collected for participant JJ, and over 175 sessions for participant EGS in open-loop analysis. Broadband data were sampled at 30,000 samples/sec from the two implanted Utah microelectrode arrays (96 electrodes each). For the finger-grid task, the single- and the multi-neuron activities were recorded from a third participant, a tetraplegic 62-year-old female human subject with a complete C3-C4 spinal cord injury (referred to as participant NS). Nine sessions of the broadband neural activity were recorded from a Utah microelectrode array implanted in the left (contralateral) PPC at the junction of the post-central and intraparietal sulci of the third participant. This region is thought to specialize in the planning and monitoring of grasping movements. The open- and closed-loop performances for participant JJ were recorded, while the presented feature extraction techniques on the recorded open-loop neural data of participants EGS and NS in the trajectory and the finger-grid tasks, respectively were recorded. The participants EGS and NS had completed their participation in the clinical trial and had had the electrodes explanted.

Data was collected while the participants performed various two-dimensional control tasks, such as a center-out grid and a finger-grid task, using standard approaches to ensure adequate and balanced statistical sampling of movement directions and velocities. In the center-out task, a cursor moves in two dimensions on a computer screen from a central target outward to one of the eight targets located around a circle, and back to the center. A trial is defined to be one trajectory, either from the central location outward to the peripheral targets, or from the peripheral targets back to the center target. In the grid task, the target appears in a random location in an 8-by-8 squared grid on the computer screen and the cursor moves starting from the old target to the newly appeared target. Cursor movement kinematics were updated every 30 ms for participant JJ and every 50 ms for participant EGS. These were sufficiently short durations to result in smooth, low-lag movements. For the purposes of this study, trajectories were extracted from 200 ms after target presentation to 100 ms before the cursor overlapped the target. This segment of time captures a window where the intent of the participant is well defined, after reacting to the presented target and before possibly slowing down as the cursor approaches the target. Neural features were regressed against cursor velocity, which, for simplicity, was modeled as constant amplitude. Each of these tasks was conducted in either open-loop, in which the cursor movements were fully generated by the computer and the participant did not directly control the position of the cursor, but instead imagines control over a visually observed, computer-controlled cursor, or closed-loop, in which the cursor movements were under the full control of the participant with no assistance from the computer.

For the finger-grid task, a text cue (e.g., ‘T’ for thumb) was displayed to the participant on a computer screen in each trial. Then, the participant immediately attempted to press the corresponding finger of the right hand. To model the multi-finger tasks, a muscle model and somatotopy open-loop muscle activation model posits that the representational structure should align with the coactivation patterns observed in muscle activity during individual finger movements. Conversely, the somatotopy model suggests that the representational structure should correspond to the spatial arrangement of the body, wherein neighboring fingers exhibit similar representation. Although somatotopy typically pertains to physical spaces resembling the body, in this context, the term broadly to encompasses encoding spaces that resemble the body.

To reduce the effect of high-frequency noise, which was not removed by the recording hardware, a common average referencing (CAR) process was applied to the recorded broadband neural data as the first step of the preprocessing. To apply the CAR, principal component analysis (PCA) was used to remove the top two principal components across each electrode before transforming the remaining principal components back to the time domain. After applying the CAR to the recorded broadband data, an 8-order elliptical high pass filter with the cut-off frequency of 80 Hz, pass-band ripple of 0.01-dB, and a stop-band attenuation of 40 dB was applied to the neural data post CAR to exclude the low frequency variations in the broadband neural activities. An 80 Hz filter was used since a window size of 30 ms used for participants JJ and Ns, and a window size of 50 ms was used for participant EGS. The 80 Hz filter is small enough to assume that the lower frequency activities are excluded from the broadband neural activity in the 30 ms and 50 ms windows. Moreover, to mitigate potential residual 60 Hz noise, a lower cutoff frequency of 80 Hz was established.

As explained above, the example feature extraction network may be trained using a two-stage optimization problem is created that transforms broadband signals into movement kinematics within a brain-machine interface cursor control paradigm. Supposing that x∈ [symbol] represents a one-dimensional feature extraction network input, which consists of S samples of the broadband neural data recorded from one electrode for one electrode, which has been sampled at the sampling frequency of F_{S} Hz. The example feature extraction network (FENet) can be represented as a function [symbol] : [symbol] → [symbol] which maps the input waveform to a M-dimensional neural feature space. M<S shows the number of extracted features and N is the number of electrodes. ψ corresponds to the feature extraction (in this case, the example FENet) parameters. The decoder can be represented by g_{θ}(.), in which g is parameterized by θ. Then, the supervised optimization problem that should be solved to find the parameters of the example feature extraction network FENet and the decoder will be as below:

ψ*,θ*=argmin_{ψ,θ}E_{(x,y)∈D} [symbol] (g_{θ}( [symbol] _{ψ}(x)),y) Equation 1

where (x, y) are the samples in the labeled dataset, D. [symbol] represents the loss function, which in the regression problem, is the mean square error between the correct and the predicted movement kinematics of the cursor velocity. According to the assumption that the generative process that produces the broadband neural activity across different channels is probabilistically ubiquitous, the example feature extraction network is designed such that it learns a single set of parameters, ψ, for all the electrodes. Thus, when the neural data recorded from N electrodes is passed to the feature extractor as an input, a similar example FENet with similar set of parameters, ψ, is applied to all the electrodes to generate the output features.

The architecture of the example FENet in the brain machine interface system is shown in the system 350 in FIG. 3C above. In this example the number of input data samples S was 30 ms of the recorded broadband neural data, which includes 900 samples for participants JJ and NS, and 50 ms of the recorded broadband neural data, which includes 1500 samples for participant EGS. The inputs were passed to the feature engineering the feature engineering modules 312, 314, and 316 to provide batch outputs.

In the experiments, other features were generated using other known feature extraction methods for comparison to the features generated by the example FENet. All features were generated in real time and from each 30 ms bin for participants JJ and NS, and from each 50 ms bin for participant EGS. To extract wavelet features (WTs) a db20 mother wavelet with 7 scales on moving windows (no overlap) of the time series recorded from each electrode was used. The db20 mother wavelet was selected as it contains filters with length 40 and can model the high pass and low pass filters of WTs more accurately compared to other Daubechies wavelet families. The mean of absolute-valued coefficients for each scale was calculated to generate M=8 time series per electrode, including seven detailed coefficients and one approximation coefficient generated by the WT high-pass filters and the final stage WT low-pass filter, respectively.

To generate threshold crossing features (TCs), the neural data was thresholded at −3.5 times the root-mean-square (RMS) of the noise of the broadband signal, independently computed for each electrode, after band-pass filtering the broadband signal between 250 Hz and 5 KHz. TCs events were counted using the same intervals as those for WTs and the example FENet. Other features extracted included Multi-Unit Activities (MUA) and High-Frequency Local Field Potentials (HFLFP).

To derive the MUA features, the raw broadband neural data underwent a bandpass filtering process with a range of 300 to 6000 Hz. Following this, customized root mean square (RMS) values were calculated to generate the MUA signal for each bin.

To generate the HFLFP features, the raw broadband neural data from each electrode underwent a second-order band-pass filtering process using a Butterworth filter with low and high cutoff frequencies set at 150 Hz and 450 Hz. The power of the filter output was then calculated and used as the HFLFP feature for each electrode. The corresponding features were concatenated together to generate a larger feature matrix that include both types of extracted features FENet-HFLFP and TCs-HPLFP.

During the open-loop and offline analysis, no form of smoothing was applied to the features under investigation since smoothing techniques have the potential to artificially enhance the performance of decoders and smoothing introduces a delay in patient control. In contrast, during closed-loop control analysis, exponential smoothing was employed as a preprocessing step for the extracted features. This was done to mitigate abrupt changes and jitters, while also introducing a latency in the control of the participant for improved stability. In the decoding pipeline, exponential smoothing was utilized through non-causal filtering to preserve causality.

Given the flexibility of the design of the example FENet and WTs to accommodate varying numbers of feature extraction levels, the resulting impact on the number of features extracted from each electrode necessitated the reduction of dimensionality. This reduction is essential to prevent overfitting of the decoder during individual sessions. To address this concern while maintaining the single channel architecture of the feature extraction technique, Partial Least Square (PLS) regression was used. Specifically, PLS regression was independently applied to the features extracted from each channel. The objective was to condense the 8 extracted features obtained from each electrode into a smaller set of features, specifically 2 features in this case.

The example feature extraction network is designed to improve the closed-loop control of external devices. One test involved the use of the test brain machine interface system such as that in FIG. 1 to manipulate a cursor as explained above. Threshold-based neural wave form crossings represent the current standard for closed-loop control and is the method that underlies best-in-world closed-loop control performance. The test participant was instructed to guide a cursor controlled by the system 100 towards visually cued targets on a computer screen. Testing was done in both a “center-out” environment, in which targets alternated between a central location and one of the eight pseudo-randomly chosen peripheral locations, or a “grid” environment, in which the target was pseudo randomly chosen from an 8-by-8 grid of targets. The data used to train the decoders mapping neural features to behavior were either collected in an open-loop setting or using interleaved blocks of closed-loop data that included use of both the example FENet-features and TCs to minimize the chances that training data would bias performance in favor of the example FENet or TCs. All experiments were double-blind using block-interleaved scheduling. A first set of trials involved open-loop center out using a training CAR and decoder for features A and B. A second set of trials was conducted using double-blind closed loop center out for retraining the decoder for features A and B. The participants were asked for their preference after the second set of trials. A third set of trials was conducted using double-blind closed loop center out for features A and B. A fourth set of trials was conducted using double-blind closed loop grid for features A and B.

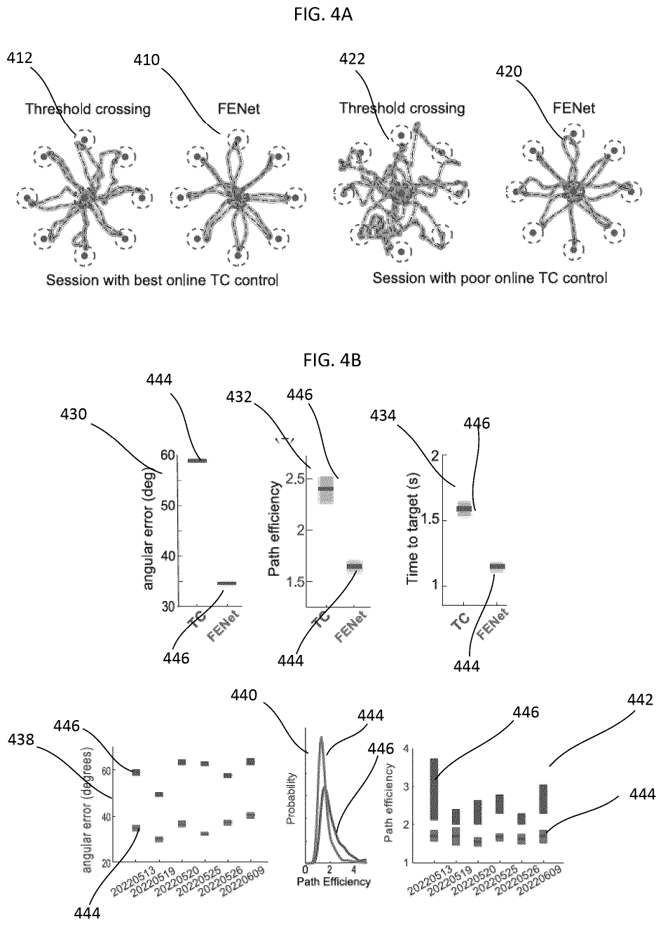

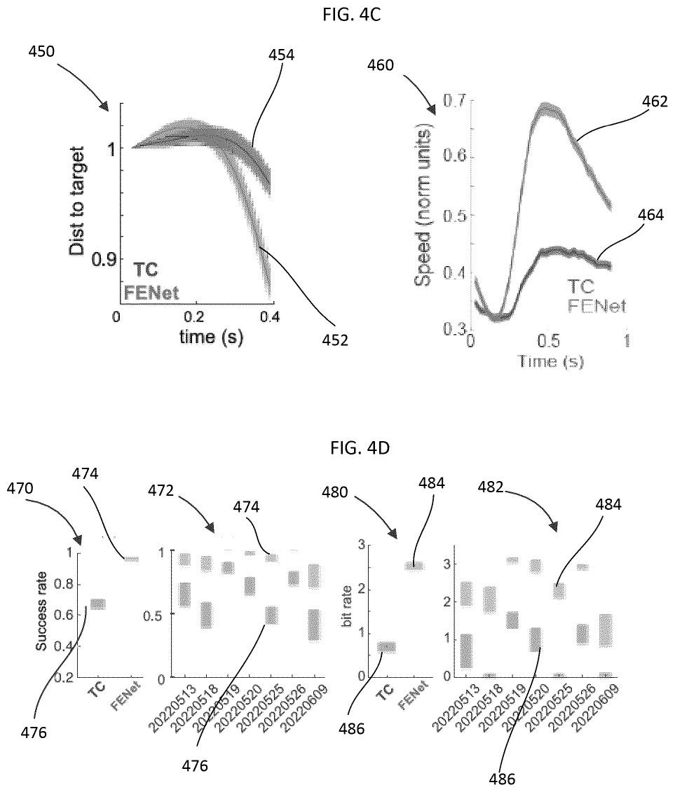

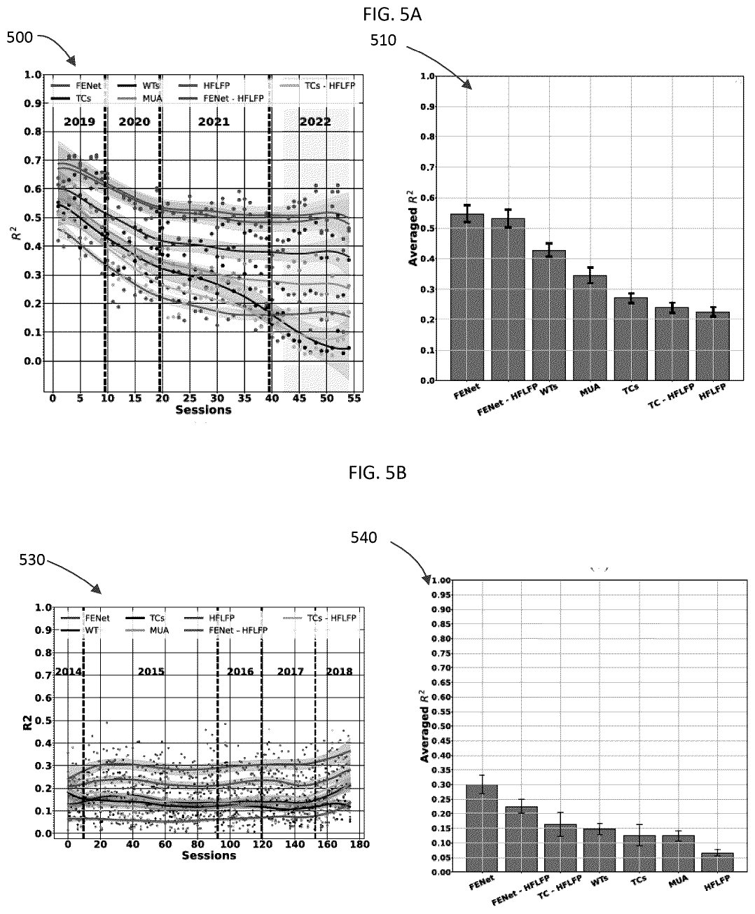

FIG. 4A shows a first graph 410 showing the test participant responses for a BMI based on the example feature extraction network that is compared with a second graph 412 that shows the responses for a BMI-controlled cursor movement using threshold-based neural waveform crossings (TCs) based features for a test participant. The graphs 410 and 412 show the results from the best on line control by the participant using the TC. FIG. 4A shows a first graph 420 showing the test participant responses for a BMI based on the example feature extraction network that is compared with a second graph 422 that shows the responses for a BMI that is based on threshold-based neural waveform crossings (TCs) for a test participant under poor on line control using the TC.

Neural decoders employing FENet-based features outperformed TC-based features across all metrics. The difference in performance is visually striking when viewing the two approaches in the interleaved block-design or when visualizing the trajectories across movements as shown in the graphs in FIG. 4A. As shown in graphs 412 and 422, there is high daily variability in control quality using TCs. TCs control quality has degraded substantially in the graph 422 while FENet has largely preserved performance as shown in graph 420.

FIG. 4B shows graphs plotting various measurements of the output of the example feature extraction network and the TC-based features for the cursor movement. A graph 430 plots instantaneous angular error, a graph 432 plots path efficiency, a graph 434 plots time to target, a graph 438 plots instantaneous angular error for different closed loop sessions, a graph 440 plots path efficiency against probability, and a graph 442 plots path efficiency for different closed loop sessions. Data bars and traces 444 represent the data from the example feature extraction network while data bars and traces 446 represent data from the known TC based feature extraction. Instantaneous angular error captures the angle between the vector pointing towards the target and the instantaneous velocity of the cursor. Path efficiency was measured as the total distance traveled end route to the target normalized by the straight-line distance from the starting location to the target. Distance to target (mean+/−95% confidence interval, CI) was used to quantify cursor responsiveness to the intent of the participant. Here, latency from target onset to goal-directed movements is shorter for FENet-based features as compared to TCs. As shown in the graphs in FIG. 4B, FENet-based features improved cursor trajectories as measured by reduced instantaneous angular error, improved path efficiency, and reduced time to target as shown in the graphs 430, 432, and 434.

Further, the example feature extraction network FENet improved the responsiveness of the cursor to the intent of the participant, decreasing the latency between target onset and the time the cursor first moved towards the target as shown in a graph 450 in FIG. 4C that plots distance to target (the averaged distance to target for center-out task) against time. A plot 452 represents the distance to target from the example feature extraction network and a plot 454 represents the distance to target from the TC based system. To account for variations in trial lengths, the graph 450 depicting the average distance to the target across multiple trials has been generated using the duration of the smallest trial as a reference. Consequently, the graph 450 does not extend in time until the target has been reached. A graph 460 shows average speed plotted against time for the example feature extraction network versus TC features. A plot 462 represents the average speed from the example feature extraction network and a plot 464 represents the average speed from the TC based system.

Success rate was also determined within an 8×8 grid task. Success was measured as the ability to move the cursor to and hold a target (0.5 second hold time) within 4 seconds. FIG. 4D shows a graph 470 that plots overall success rate and a graph 472 that plots success rate for individual closed loop sessions within an 8 by 8 grid task over a few months. Bars 474 represent the success rates for the example feature extraction network and bars 476 represent the success rates for the TC based system. FIG. 4F shows a graph 480 that plots overall bit rate (rate of hitting targets during trials) and a graph 482 that plots bit rate for individual closed loop sessions within the 8 by 8 grid task. Bars 484 represent the bit rates for the example feature extraction network and bars 486 represent the bit rates for the TC based system. Success was measured as the ability to move the cursor to and hold a target (0.5 second hold time) within 4 seconds. Improvements on both fronts resulted in substantial improvements in overall task performance during the grid-task, including success-rate and bitrate as shown in the graphs 460, 470, 472, 480 and 482. As part of the double-blind experimental design, the participant was asked which of the two methods was preferred. In every instance, the participant reported a strong preference for the FENet-based decoder.

Baseline performance with TCs was poor during testing as the consequence of significant degradation in the quality of the neural signals over the lifetime of the recording arrays. The example feature extraction network FENet improves the performance across the lifetime of the array (even when TCs produce excellent performance) and across the participants.

Direct comparison in closed-loop testing is ideal but opportunities for such testing are relatively limited. To increase the scope of comparison across time and feature extraction techniques, the ability of the example feature extraction network FENet to reconstruct the movement kinematics was evaluated using previously collected neural data recorded from implanted electrode arrays. In particular, data collected during an “open-loop” paradigm was used, in which the participant attempted movements as cued by a computer-controlled cursor performing the center-out task. Given that neural networks have the potential to overfit, the data used to train the example FENet was 100% separate from the validation and the test data.

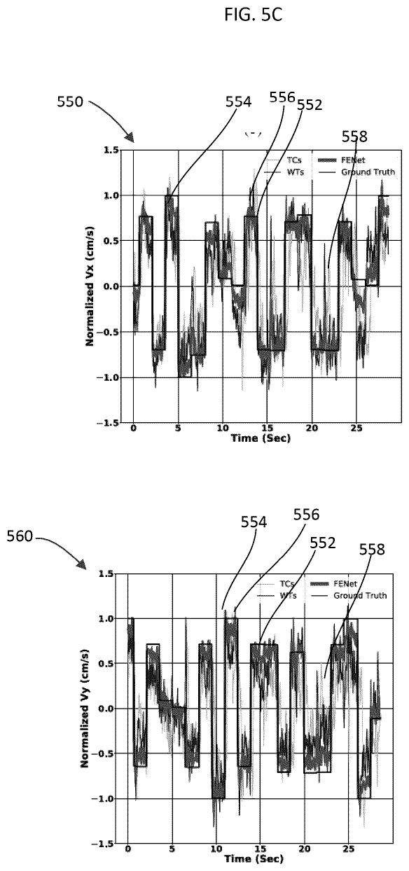

FIG. 5A shows a graph 500 that shows cross-validated coefficient of determination, R² against sessions over time and a graph 510 that shows averaged coefficient of determination for the research participant JJ over 54 recorded sessions spanning from 2019 to 2022. The shaded regions in the graph 500 shows the closed-loop sessions. The band in each time series shows the range of its 95% confidence interval of a LOESS fit. The graphs 500 and 510 show the reconstruction performance of a linear decoder operating on TCs, WTs, and FENet extracted features. The graphs 500 and 510 also compares the performance of the example FENet with other types of feature extraction methods, including Multi-Unit Activities (MUA), High-Frequency Local Field Potentials (HFLFP), and the combination of FENet and TCs with HFLFP. FIG. 5B shows graphs 530 and 540 also compares the performance of the example FENet with TC, WT, Multi-Unit Activities (MUA), High-Frequency Local Field Potentials (HFLFP), and the combination of FENet and TCs with HFLFP on the research participant EGS over 175 recorded sessions spanning from 2014 to 2018. The feature decoding stage was held constant across all feature extraction techniques so as to minimize confounds to interpretation.

FIG. 5C shows a graph 550 of normalized velocity in the x direction and a graph 560 of normalized velocity in the y direction for the example FENet against TCs, WTs, and ground truth. The plots for the graphs 550 and 560 were obtained in a single experimental session 20190507 of reconstructed instantaneous velocity of participant JJ showing reconstructions from FENet, WTs, and TCs. The line 552 shows the ground-truth target velocity, and the lines 554, 556, and 558 show the reconstruction of the feature extraction techniques from FENet, WT, and TCs respectively.

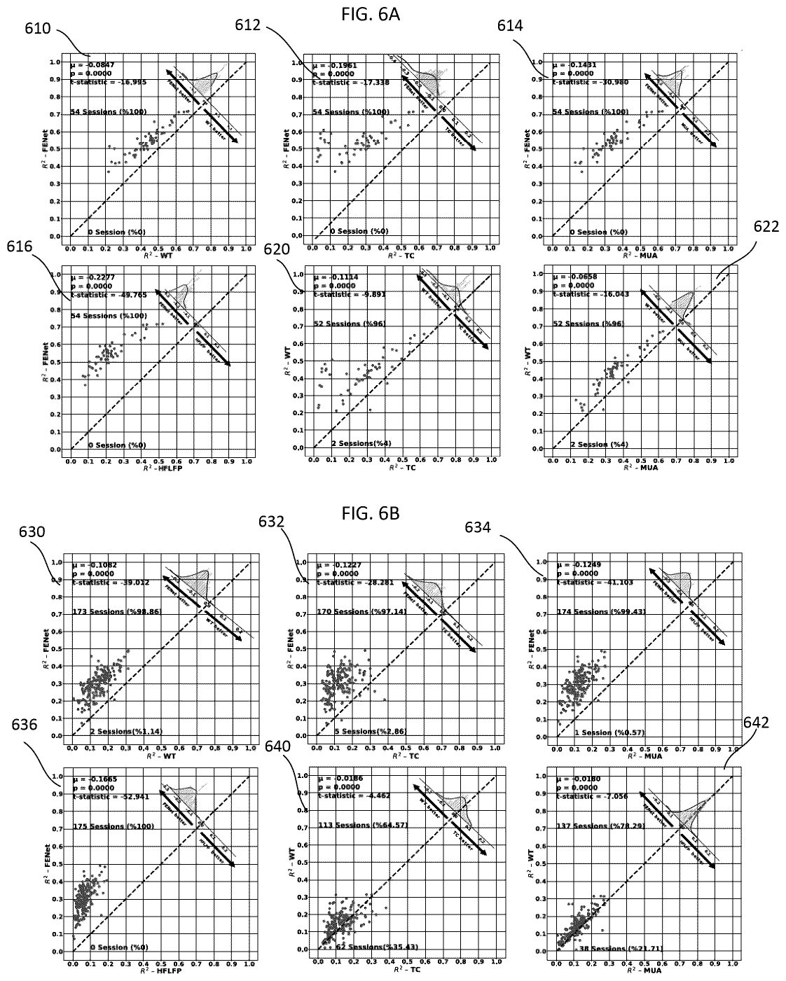

FIG. 6A shows graphs 610, 612, 614, and 616 that plot the cross-validated coefficient of determination, R², for the example feature extraction network against the respective cross-validated coefficient of determination, R², for WT, TC, MUA, and HFLFP respectively for the participant JJ. Graphs 620 and 622 plot the cross-validated coefficient of determination, R² for WT against the cross-validated coefficient of determination, R² for TC and MUA respectively for the participant JJ. FIG. 6B shows graphs 630, 632, 634, and 636 that plot cross-validated coefficient of determination, R² for the example feature extraction network against the respective cross-validated coefficient of determination, R² for WT, TC, MUA, and HFLFP respectively for the participant EGS. Graphs 640 and 642 plot the cross-validated coefficient of determination, R² for WT against the cross-validated coefficient of determination, R² for TC and MUA respectively for the participant EGS.

The graphs in FIGS. 6A and 6B show the comparison of the cross-validated coefficient of determination R² of a linear decoder operating on one feature extraction technique versus the other feature extraction technique for participant JJ, and participant EGS. The dots on the graphs show the sessions. The dashed line shows y=x. The percentage of dots on each side of y=x shows the number of sessions in favor of the corresponding feature extraction technique. The t-test statistics have been calculated to show the confidence level of the reported statistics. According to these figures, a linear decoder operating on the FENet-based features provides superior performance in terms of R² compared to other feature extraction techniques for both human participants.

The graph 510 in FIG. 5A and the graphs 610, 612, 614, 616, 620 and 621 in FIG. 6A show that for participant JJ, the example FENet improved the average cross-validated coefficient of determination (R²) of TCs (t=−17.338, p=0.0000) and WTs (t=−19.368, p=0.0000) from 0.27 and 0.43 to 0.55, respectively. The graph 540 in FIG. 5B and the graphs 630, 632, 634, 636, 640, and 642 in FIG. 6B show that for participant EGS, the example FENet improves the average cross-validated R² value of TCs (t=−39.012, p=0.0000) and WTs (t=−28.281, p=0.0000) from 0.13 and 0.15 to 0.30, respectively. The graph 500 in FIG. 5A, the graph 520 in FIG. 5B and the graphs in FIGS. 6A-6B show that these improvements were found for each individual recording session as well. The graphs 550 and 560 in FIG. 5C show example reconstructions of the cursor velocity in X and Y directions for a session recorded from JJ in 2019 and highlights how FENet both reduces trial-to-trial variability (FENet in the plots 554 is closer to ground truth 552 for each trial repetition) and within-trial variability (FENet in the plots 554 demonstrates less variability within each trial).

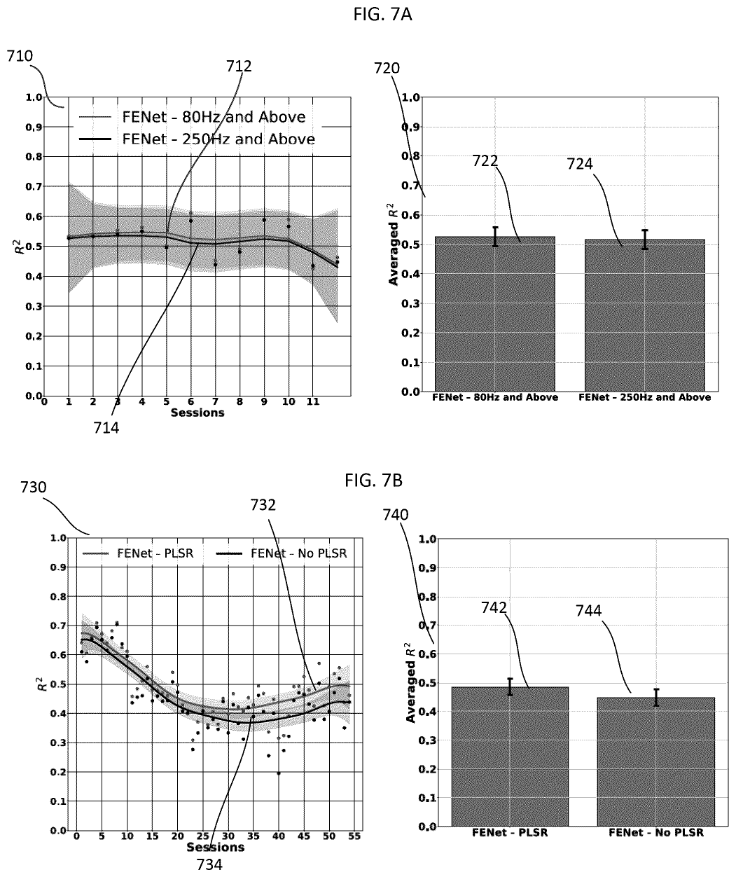

To examine if the example FENet is using local field potential (LFP) for its long-term stability, the broadband data recorded from the closed-loop sessions was filtered before extracting the FENet features by using the high pass filters with the cutoff frequency of 80 Hz and 250 Hz, respectively. An 80 Hz filter was used since window size of 30 ms used for participant JJ is small enough to assume that the lower frequency activities are excluded from the broadband neural activity in the 30 ms window. Moreover, to mitigate potential residual 60 Hz noise, a lower cutoff frequency of 80 Hz was established. FIG. 7A shows a graph 710 having a plot 712 representing a cutoff frequency of 80 Hz and a plot 714 representing a cutoff frequency of 250 Hz. A graph 720 shows the averaged coefficient of determination for a cutoff frequency of 80 Hz in a bar 722 and a cutoff frequency of 250 Hz in a bar 724. The performance of a linear decoder operating on the example FENet slightly drops per session as shown in the graph 710 and on average as shown in the graph 720 when data is filtered using a high-pass filter with the cut-off frequency of 250 Hz compared to the case that the cut-off frequency of the high-pass filter is 80 Hz. This shows the example FENet is not directly affected by the information that is extracted from the LFP band.

A comprehensive evaluation was conducted of the effect of a partial least-squares regressor (PLSR) on the performance of a linear decoder operating on the example feature extraction network (FENet) using all 54 sessions of participant JJ. The performance of the example feature extraction network was compared with and without Partial Least Squares Regression (PLSR) applied to the top 40 electrodes in these sessions. The top 40 electrodes were selected to mitigate overfitting in the linear decoder, particularly in cases where PLSR is not applied. FIG. 7B shows a graph 730 having a plot 732 representing the example FENet with a PLSR and a plot 734 representing the example FENet without a PLSR. A graph 740 shows the averaged coefficient of determination for the example FENet with a PLSR in a bar 742 and the example FENet without a PLSR in a bar 744. The results presented in the graphs 730 and 740 demonstrate that the example FENet, regardless of PLSR, effectively captures informative features from the broad neural data. The application of PLSR serves to reduce feature dimensionality and prevent overfitting of the decoders when working with limited neural data from human participants per session. The band in each time series shows the range of its 95% confidence interval of a LOESS fit. The example feature extraction network does not rely on the low-frequency (<250 Hz) local-field potentials to achieve its enhanced decoding performance.

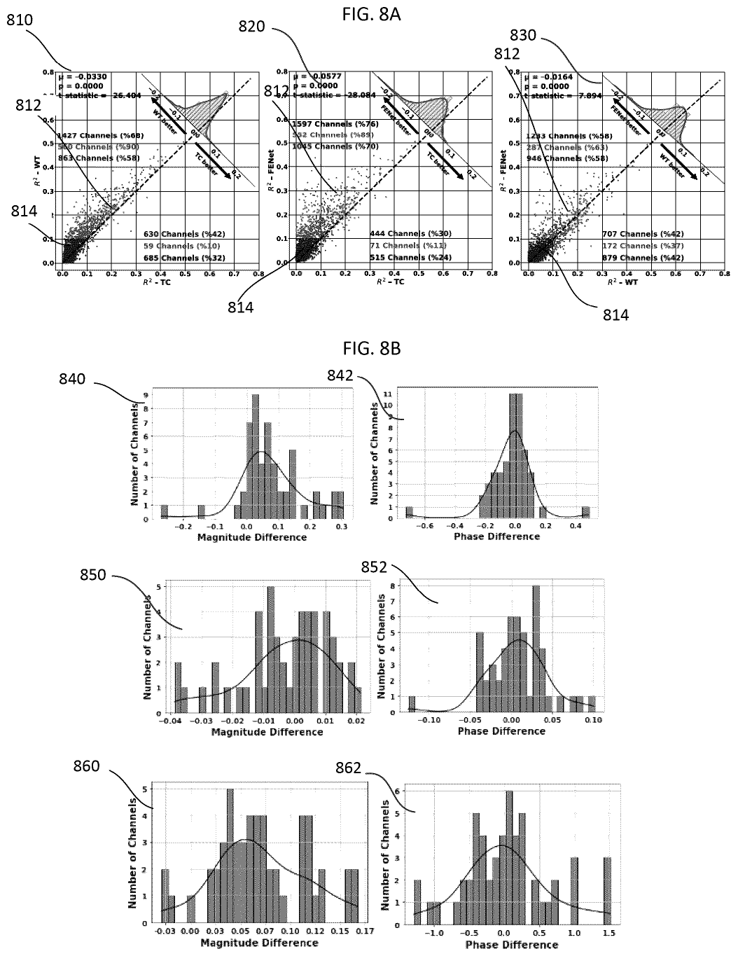

Open-loop single-electrode performance of a linear decoder operating on FENet, WTs, and TCs was examined. A comparison was made of the cross-validated coefficient of determination, R², of linear decoders for the example FENet, WTs, and TCs as different feature extraction techniques on all 192 neural channels (electrodes) of 2019 sessions of participant JJ. FIG. 8A shows a graph 810 that plots cross-validated coefficient of determination the channels (electrodes) as dots in relation to feature extraction of WTs and TCs. A graph 820 plots cross-validated coefficient of determination the channels (electrodes) as dots in relation to feature extraction of the example FENet and TCs. A graph 830 plots cross-validated coefficient of determination the channels (electrodes) as dots in relation to feature extraction of the example FENet and WTs. In the graphs 810, 820 and 830, the dashed line shows line y=x. A cluster of dots 812 show the electrodes with R² greater than 0.1 in at least one of the feature extraction techniques and a cluster of dots 814 are the electrodes with R² smaller than 0.1 for both techniques. Analysis is performed on electrodes that carry more information about movement kinematics. The reported bottom, middle, and top numbers in the graphs 810, 820 and 830 demonstrate the percentage of electrodes in each side of y=x for all the channels, channels that carry more information (R² greater than 0.1), and channels that carry less information (R² smaller than 0.1), respectively. The percentage of dots on each side of the line y=x shows the number of electrodes in favor of the corresponding feature extraction technique. The t-test statistics have also been reported to show the confidence level of the reported statistics. According to this analysis, the example FENet-based features improve the decoding performance of each single electrode in terms of R² compared to TCs and WTs.

To compare the preferred direction and tuning properties of the same electrode in two feature extraction techniques, a linear decoder was trained on a feature that was extracted from that similar electrode for each feature, and the magnitude and angle difference between the vectors that are generated by the coefficients of the trained linear decoders was plotted. FIG. 8B shows graphs 840, 850 and 860 that show the magnitude difference against the number of channels. Graphs 842, 852, and 862 show the phase difference (shown in radian) against the number of channels. The parameters of linear tuning models for the same electrode were compared between two features. Although the feature extraction techniques are inherently different, activity of a similar electrode maintains its preferred direction independent from a specific feature extraction technique. The phase difference is shown in radian. Graphs 840 and 842 show the differences in magnitude and phase for an electrode for the example FENet compared with TCs. Graphs 850 and 852 show the differences in magnitude and phase for an electrode for the example FENet in comparison to WTs. Graphs 860 and 862 show the differences in magnitude and phase for an electrode for the WTs in comparison with TCs. As designed, the example FENet improves population decoding by increasing the behavioral information content of almost every electrode. Although FENet improves the coefficient of determination between neural features and kinematics compared to WTs and TCs, the graphs in FIG. 8B show that the example FENet reports similar tuning preferences to TCs and WTs at the same electrodes.

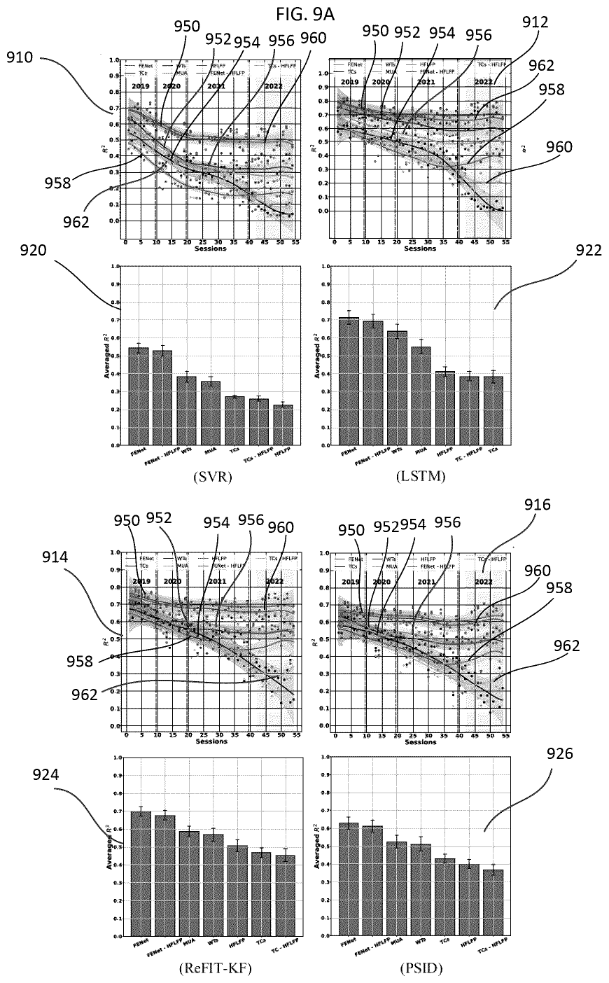

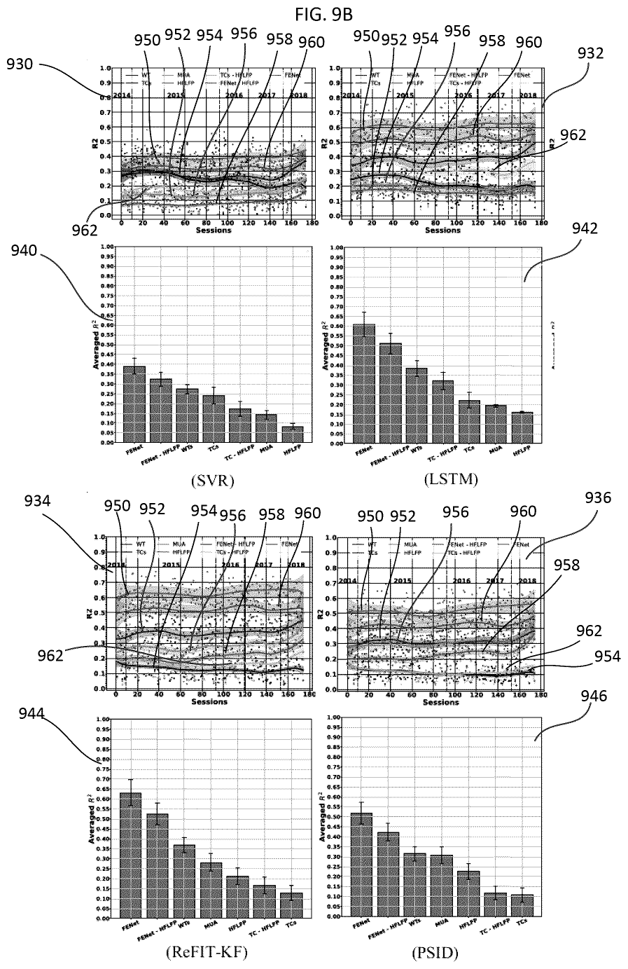

To ensure that improvements in the example feature extraction method generalize across feature decoding methods, the performance of additional feature decoders, namely Support Vector Regression (SVR), Long-Short Term Recurrent Neural Network (LSTM), Recalibrated Feedback Intention-Trained Kalman filter (ReFIT-KF), and Preferential Subspace Identification (PSID) were compared. Open-loop performance evaluation of additional decoders employing diverse feature extraction techniques. FIG. 9A shows a first set of graphs 910, 912, 914, and 916 that plot coefficient of determination against sessions over different years for the participant JJ using the SVR, LSTM, ReFIT-KF, and PSID decoders respectively for a center out task. A second set of graphs 920, 922, 924, and 926 that plot averaged coefficient of determination against different features for the participant JJ using the SVR, LSTM, ReFIT-KF, and PSID decoders respectively for the center out task.

FIG. 9B shows a first set of graphs 930, 932, 934, and 936 that plot coefficient of determination against sessions over different years for the participant EGS using the SVR, LSTV1, ReFIT-KF, and PSID decoders respectively. A second set of graphs 940, 942, 944, and 946 that plot averaged coefficient of determination against different features for the participant EGS using the SVR, LSTM, ReFIT-KF, and PSID decoders respectively.

The analyzed feature extraction techniques include FENet (plot 950), Wavelet Transform with db20 mother wavelet (WTs) (plot 952), Threshold Crossings (TCs) (plot 954), Multi-Unit Activity (MUA) (plot 956), High-Frequency Local Field Potentials (HFLFP) (plot 958), the combination of FENet and HFLFP (plot 960), and the combination of TCs and HFLFP (plot 962) in the graphs 910, 912, 914, 916, 930, 932, 934, and 936. In this test, the example FENet was trained on center-out task data from participant JJ using a linear decoder and kept unchanged during decoder training. All the decoders consistently outperformed other feature extraction techniques when operating with the example FENet.

The data shown in the graphs in FIGS. 9A and 9B provide a comprehensive evaluation of the performance of these decoders operating on different feature extraction techniques. As shown in the graphs in FIGS. 9A and 9B, the example FENet improves the coefficient of determination, R², of the decoding compared to the other feature extraction techniques for all the presented decoders.

The open-loop results with the example FENet were evaluated using neural data and behavior binned at fine temporal resolution (30 ms bins) and without smoothing the extracted features. This was motivated by the desire for the example FENet to be maximally useful for closed-loop control where smoothing decreases the responsiveness of the closed-loop system by using potentially outdated neural information. However, recognizing that the example FENet could also be used for slow-timescale applications, the example FENet was tested on how it performed against TCs when smoothing the extracted features by extracting the features from a larger window size.

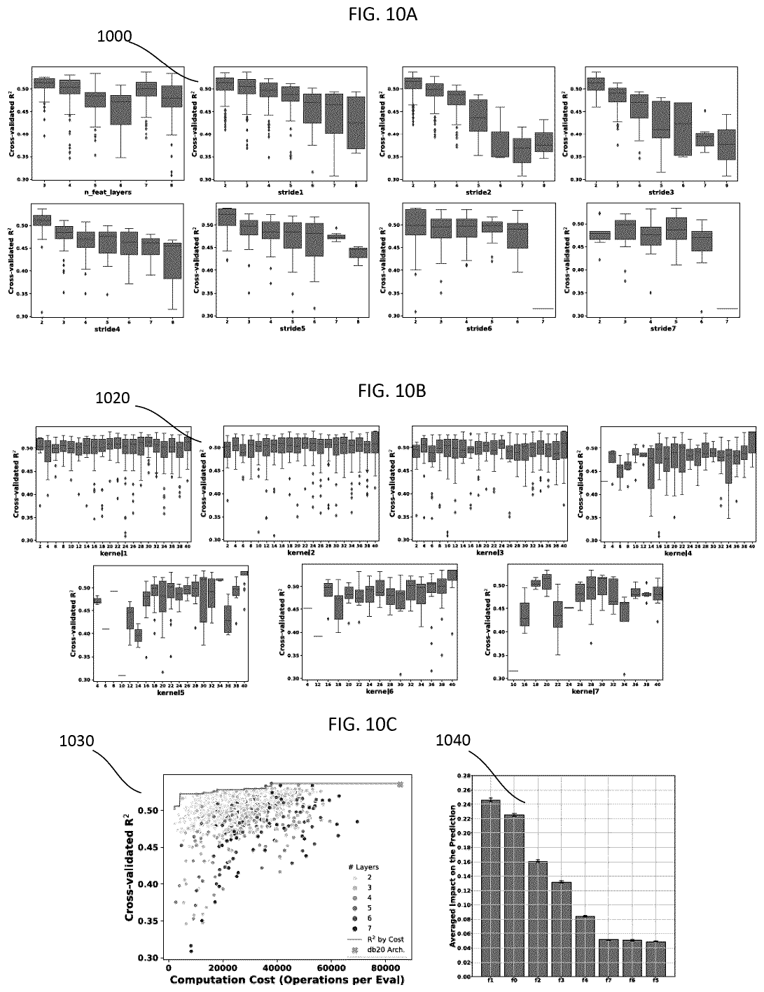

Parameter sweeps using Bayesian optimization on the example FENet model were conducted to assess the importance and impact of each hyperparameter in the architecture. FIG. 10A is a series of graphs 1000 showing different strides of the filter in relation to cross validated coefficient of determination for the example feature extraction network. FIG. 10B is a series of graphs 1020 showing different kernel sizes of the filter in relation to cross validated coefficient of determination for the example feature extraction network. The results indicate a correlation between the values of the linear decoder and the parameter values. Notably, the strides of the initial layers emerge as the most influential parameters, with smaller strides yielding higher performance. This is because smaller strides allow the convolutional kernels to cover a greater variety of patterns in the input. Conversely, larger strides limit the coverage between consecutive kernel movements, resulting in the filters learning fewer patterns. Additionally, the kernel size becomes more crucial in later layers compared to the initial layers. This suggests that the inputs to later layers summarize information from multiple samples in the preceding layers. Consequently, the network becomes more sensitive to kernel size when combining richer features with different kernel sizes, as these layers combine samples providing less abstract information than deeper layers. FIG. 10C shows a graph 1030 that plots the cross validated coefficient of determination against the computational costs for different numbers of layers in the example feature extraction network. The cross-validated coefficients of determination were measured from a linear decoder operating on features extracted by using different FENet architectures and the associated computational cost of these different architectures was measured. The proportional Shapley values of all the electrodes were averaged over all the sessions for participant JJ. FIGS. 10A and 10B show the robustness of the example FENet against the change in the window size used to update the feature extraction process in our trajectory tasks.

To assess and understand the effectiveness of the extracted features obtained through diverse feature extraction techniques, a rigorous analysis using offline data from a specific session labeled as 20210312 was conducted. The offline data of the sample session 20210312 was partitioned into eight center-out task trials, each trial corresponding to a different target. The target was named with x>0 and y=0 as Target0. Subsequently, the feature values of the first and the second top electrodes of this session were averaged across all trials.

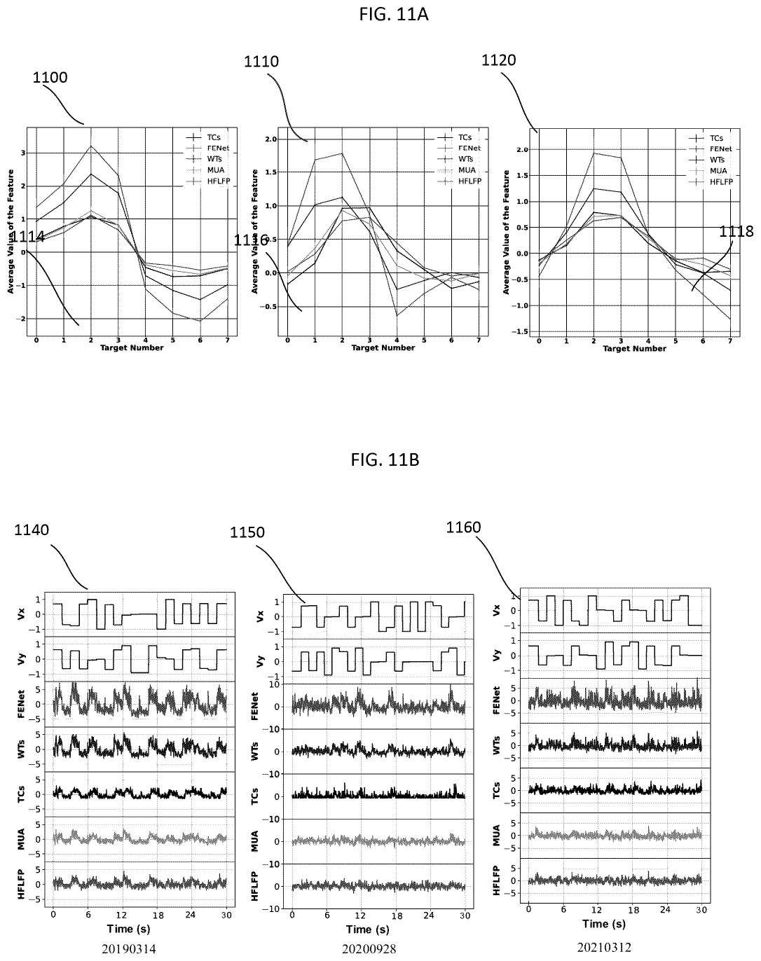

FIG. 11A shows a graph 1100 showing the average value of the feature determined from the first electrode by the example FENet in comparison with that determined by TCs, WTs, MUA, and HFLFP for a set of seven different targets during a first session. A graph 1110 shows the average value of the feature determined from the first electrode by the example FENet in comparison with that determined by TCs, WTs, MUA, and HFLFP for a set of seven different targets during a second session. A graph 1120 shows the average value of the feature determined from the first electrode by the example FENet in comparison with that determined by TCs, WTs, MUA, and HFLFP for a set of seven different targets during a third session. FIG. 11B shows a graph 1140 showing the actual values of the features during the initial 30 seconds of the recorded data determined from the first electrode by the example FENet in comparison with that determined by TCs, WTs, MUA, and HFLFP for a set of seven different targets in the first session. A graph 1150 showing the actual values of the features during the initial 30 seconds of the recorded data determined from the first electrode by the example FENet in comparison with that determined by TCs, WTs, MUA, and HFLFP for a set of seven different targets in the second session. A graph 1160 showing the actual values of the features during the initial 30 seconds of the recorded data determined from the first electrode by the example FENet in comparison with that determined by TCs, WTs, MUA, and HFLFP for a set of seven different targets in the third session. The results demonstrate the preservation of the fundamental tuning characteristics of the neurons, albeit with an enhanced Signal-to-Noise Ratio (SNR) and better trial separability for the example FENet. Notably, the example FENet exhibited significant improvements in terms of the SNR and the ability to distinguish individual trials. These findings indicate that the example FENet provides a more robust and distinctive representation of the neural activity, thereby enhancing the performance of decoding neural signals.

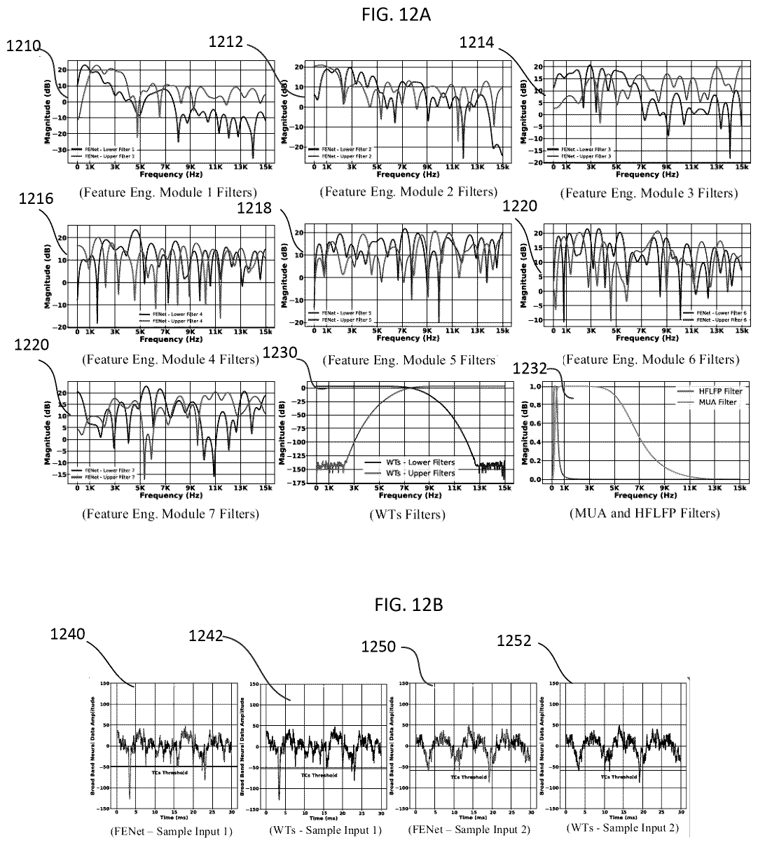

The comparative effectiveness of the trained convolutional filters of the example feature extraction network was investigated in relation to the conventional filters used for extracting WTs, MUA, and HFLFP features. Specifically, the gain, or the amplification capability, of the sample set of FENet trained convolutional filters across seven feature engineering modules was examined. FIG. 12A shows graphs 1210, 1212, 1214, 1216, 1218, 1220, and 1222 that plot the gain of the upper and lower convolutional filters of seven sample feature engineering modules of the example FENet using data from the participant JJ. A graph 1230 show the gains for the upper and lower filters of WTs using data from the participant JJ. A graph 1232 shows the gains of MUA and HFLFP using data from the participant JJ.

In contrast to the other filters, the example FENet displayed a unique characteristic of dynamically amplifying specific frequency bands during its training process. The training mechanism of the example feature extraction network takes into account the encoded information within each frequency band, allowing it to selectively enhance relevant features within different frequency ranges. This ability to dynamically amplify distinct frequency bands sets the example FENet apart from conventional feature extraction methods such as WTs, MUA, and HFLFP. By adaptively adjusting its filters based on the specific frequency information, the example FENet exhibits a more nuanced and refined approach to feature extraction, leading to improved performance in analyzing neural data.

In order to gain insights into the specific regions of input data that receive more attention from the example FENet during the prediction process, two illustrative examples of single electrode input samples were obtained from FENet and WTs were examined. These samples were collected during a specific session identified as 20190625 for the participant JJ. FIG. 12B shows a graph 1240 that plots the broadband neural data amplitude for a first sample input to the example FENet. A graph 1242 plots the broadband neural data amplitude for the first sample input to the WTs system. A graph 1250 plots the broadband neural data amplitude for a second sample input to the example FENet. A graph 1252 plots the broadband neural data amplitude for the second sample input to the WTs system.

To highlight the segments of higher importance in the predictions made by the linear decoder, pattern-coded visual representations were used in the graphs 1240, 1242, 1250, and 1252 to show the relevant sections. To accurately depict the most relevant sections of the input signals, the average Shapley value was calculated across all samples. Subsequently, the samples whose Shapley values surpassed this calculated average threshold were selectively patterned. Additionally, a horizontal line is included in the graphs 1240, 1242, 1250, and 1252 to denote the threshold utilized for extracting features associated with Threshold Crossings (TCs) from each input sample.

FIGS. 12A-12B provide evidence showing that the example FENet, following its training, leverages not only spike information (TCs) and Wavelet Transforms (WTs), but also exhibits superior capabilities in identifying local patterns within the input data. Furthermore, the example FENet demonstrates an exceptional proficiency in accurately tracking rapid and abrupt changes present in the input signals when compared to WTs. These empirical findings collectively show the example FENet possesses the ability to capture more intricate and localized information, thereby resulting in enhanced feature extraction capabilities when compared to the conventional WTs and TCs approaches.

The example feature extraction network works across patients, in any implanted region of the brain, for any subset of electrodes, and for the duration of the implant recordings. Although the example FENet was trained using a particular set of patients and brain areas, the resulting solution should apply more generally to any situation in which the functional state of the brain must be inferred from electrical recordings. To show how well the example FENet generalizes to the novel data, training data were split in various ways (by time, brain area, patient, and electrode subset) and performance was compared within and across the data splits.

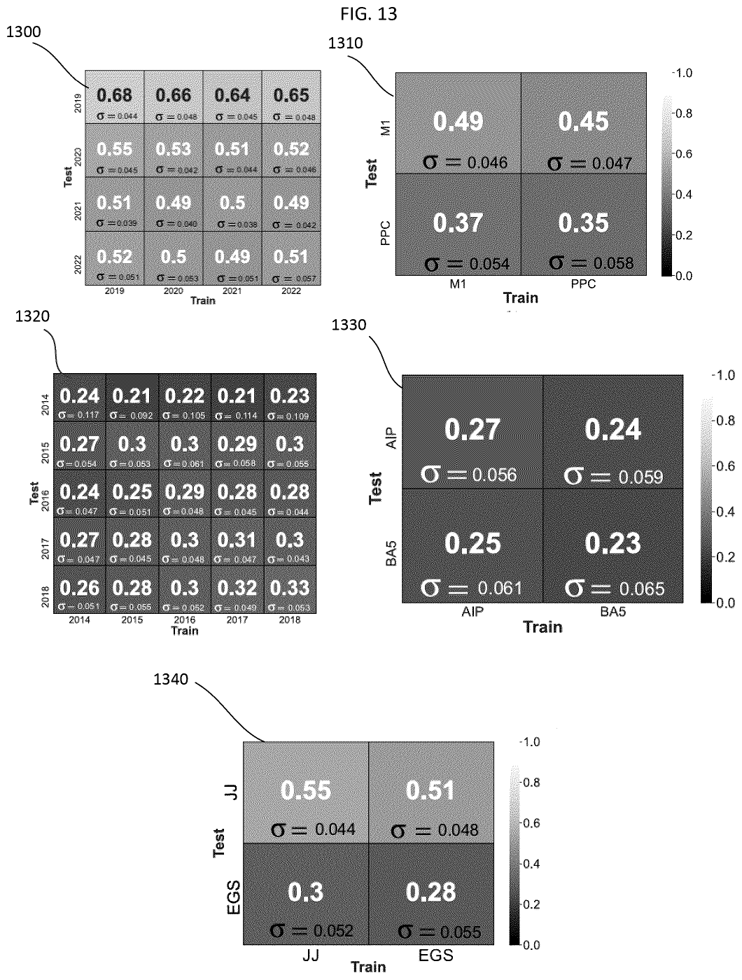

FIG. 13 shows a first chart 1300 of decode performance of the participant JJ over different time periods using coefficient of determination of training and testing and a standard deviation a for the example FENet. A chart 1310 shows the coefficients of determination on different brain regions and standard deviation for the participant JJ. A chart 1320 shows decode performance and standard deviation of the participant EGS in 2022 over different time periods using coefficient of determination of training and testing the example FENet. A chart 1330 shows the coefficients of determination on different brain regions and standard deviation for the participant EGS. In each square of the charts 1300, 1310, 1320 and 1330, the example FENet is trained on the data coming from either the sessions/region corresponding to a specific year/brain region and is tested on the other year/region to evaluate generalizability of the FENet using the center-out task. A chart 1350 shows the example FENet trained on one human subject (participant JJ) and tested on the other human subject (participant EGS).

The charts in FIG. 13 show that the example FENet generalizes within and across splits of data. For example, the chart 1300 shows decode performance on data collected from participant JJ in 2022 is similar whether the example FENet was trained on the same 2022 data or data from any previous year of the implant. This is remarkable given the significant changes in the quality of electrical recordings over this time span. Importantly, in all cases, generalization performance was significantly better than TCs or WTs applied to the same dataset as shown in the graph 510 in FIG. 5A and the graph 540 in FIG. 5B. These results show that FENet can generalize across different time periods, brain areas, patients, and electrodes as will be explained below.

The example FENet significantly improved the ability to decode instantaneous cursor velocity in the center-out and grid trajectory tasks. The example FENet could serve as a drop-in solution to improve the information content of neural features in a different task. This may be shown by the application of the example FENet to a previously published “Finger flexion grid” task dataset based on the three characteristics of the dataset.

First, intended BMI movements may be confounded with overt movements (e.g., of the head and eyes) as the participant orients to a target. The finger-grid task explicitly dissociates overt movements from the neural signals of interest by randomizing the cue location. Second, the populations of the sorted units collected during the finger-grid task exhibited representational structure that dynamically changed through time. The ability of the example FENet to recapitulate these representational dynamics, with improved signal-to-noise ratio, would further validate that FENet can be dropped into any neuroscience and neuroengineering processing chains. Third, in the finger-grid task, the ability to decode movements of each finger was tested, which demonstrates that FENet generalizes to additional variables of interest to neural prosthetics. Finally, the finger-grid dataset was collected from the participant NS, and thus, the successful application of the example FENet demonstrates generalization of the example FENet to a new participant.

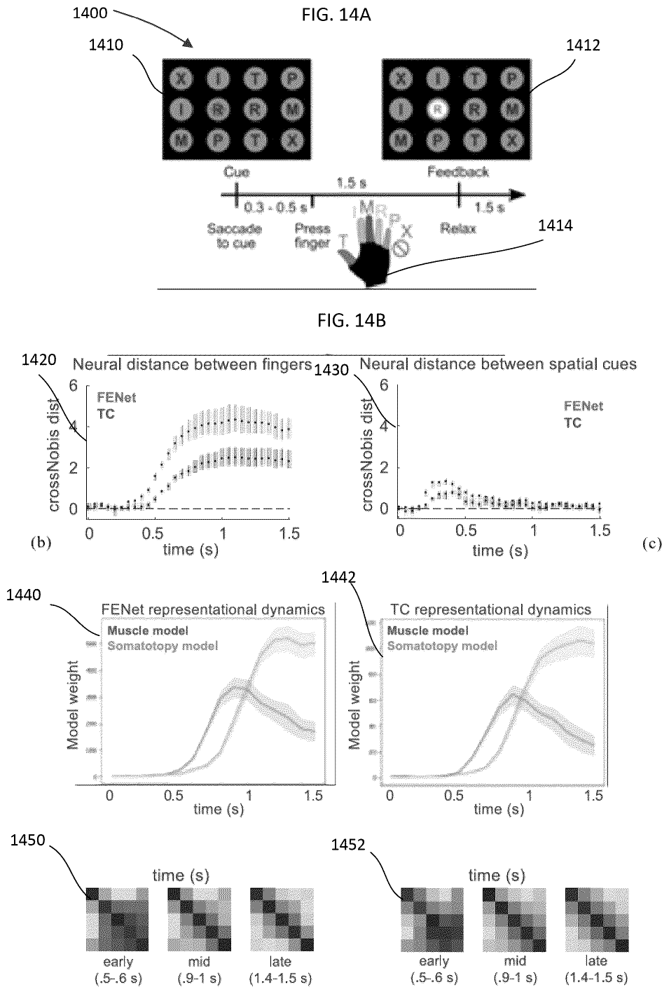

FIG. 14A is a finger flexion grid task test system 1400 that demonstrated robust brain machine interface control of individual fingers. The system 1400 includes displaying a cue image 1410 and a feedback image 1412 that reflect operations controlled by movements of a cursor by a hand 1414 of the participant. When a letter was cued by the red crosshair on the cue image 1410, the participant looked at the cue and immediately attempted to flex the corresponding finger of her right (contralateral) hand 414. A null condition ‘X’ was included, during which the participant looked at the target but did not move her fingers. The visual feedback image 1412 indicated the decoded finger 1.5 s after cue presentation. To randomize the gaze location, cues were located on a grid (three rows, four columns) in a pseudorandom order. The red crosshair was jittered to minimize visual occlusion.

FIG. 14B shows a graph 1420 that plots neural distance (Mahalonobis (crossNobis)) between fingers in time for FENet and TCs. A graph 1430 plots neural distance between targets in time for FENet and TCs. A graph 1440 plots the representational dynamics analysis (RDA) of the measured representational dissimilarity matrix (RDM) over time to show an early fit to the muscle model and a late fit to the somatotopy model for the example FENet. A graph 1442 plots the RDA of the measured RDM over time for the TCs. Confidence intervals indicate±standard error of the mean (SEM) bootstrapped across 10 sessions. The shaded regions indicate the approximate onset time of the saccade to cue (interquartile range across trials). Difference in model start time (170 ms) was significant (p=0.002, two-sided Wilcoxon signed rank test). RDM snapshots 1450 and 1452 (each max-scaled to [0, 1]) intuitively visualize the change in representational structure over time from muscle-like to somatotopic for each finger. The RDM snapshot 1450 shows the responses of the test from the example FENet. The RDM snapshot 1452 shows the response the test from the TC technique.

In response to a visual cue, the participant NS immediately attempted to press the corresponding finger, as though striking the key to a keyboard. Movements were cued by having a cursor move randomly across a 4-by-3 grid of letters. The participant oriented her head and eyes to each position on the board after which she attempted the instructed movement. The graphs 1420 and 1430 show that the example FENet features improved the ability to distinguish individual finger movements, here captured as the cross-validated Mahalonobis (crossNobis) distance between fingers. Importantly, the relative magnitude and timing of FENet encoding of the location of the spatial cue as shown in the graph 1430 was much smaller than what was found for digit encoding as shown in the graph 1420. This suggests that features produced by the example FENet are not unduly influenced by factors associated with overt movements such as head or cue position, and instead maintain the specificity of populations of sorted neurons. Finally, a comparison of graphs 1450 and 1450 in FIG. 14B shows that the example FENet preserves the representational structure and dynamics of populations of sorted neurons. Taken together, these results demonstrate that the example FENet improves decoding of a novel dataset from a new participant with electrodes implanted in different brain regions, while maintaining the specificity and preserving the detailed representational structure of sorted single neurons.

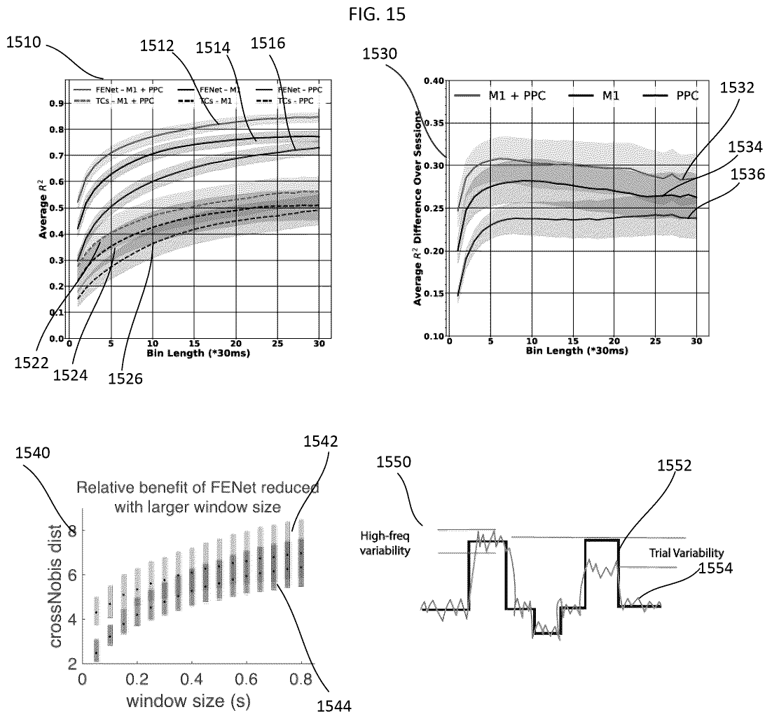

Similar to the case of the cursor control task as explained above, the performance of the example FENet against TCs was tested when smoothing the extracted features. The performance of the example FENet was robust against the change of the recording window size length in the center-out trajectory task. FIG. 15 shows a graph 1510 the averaged coefficient of determination, R² against the bin length for a linear decoder operating on FENet and TCs when the feature extraction window size is increased, which has the smoothing effect on the extracted features. A set of solid curves 1512, 1514, and 1516 show the performances for the example FENet and a set of dashed curves 1522, 1524, and 1526 show the performance for the TCs. The curves 1516 and 1526 show the performance of the feature extraction techniques on the neural features extracted from the neural data recorded from all the electrodes. The curves 1512 and 1524 show the performance of the feature extraction techniques on the neural features extracted from the neural data recorded from electrodes implanted on the Motor Cortex (M1). The curves 1514 and 1524 show the performance of the feature extraction techniques on the neural features extracted from the neural data recorded from the electrodes implanted on the Posterior Parietal Cortex (PPC). The example FENet maintains its superior performance over TCs when the feature extraction window size is increased in the trajectory task. A graph 1530 shows the average difference between the curves in the graph 1510. A plot 1532 shows the combination difference of M1 and PPC, a plot 1534 shows the average difference for M1 electrodes and a plot 1536 shows the average difference for PPC electrodes. The band in each time series shows the range of its 95% confidence interval of a LOESS fit.

A graph 1540 plots crossNobis distance against window size. A set of plots 1542 shows the crossNobis distance from the example FENet and a set of plots 1544 shows the cross Nobis distance from sorted units. This reflects measurements how the crossNobis distance metric compared between sorted neurons and the example FENet as a function of window size. At small window sizes (e.g., 50 ms) comparable benefits of the example FENet are seen over sorted units. However, as the size the window increases, the relative benefit of the FENet is reduced. A graph 1550 shows the explanation of the high frequency and the between trial variability of the kinematic prediction. A curve 1552 shows the ground-truth movement kinematics, and a curve 1554 shows the decoder prediction. The graph 1550 shows that the relative benefit of FENet is diminished with increasing smoothing windows, although it maintains a benefit over TCs.

The example feature extraction network based brain machine interface system 350 shown in FIG. 3C was trained as detailed below. To update the network parameters during training, one training session was selected and a batch of the associated broadband activities was passed to the example FENet to extract neural features. According to experiments, the best performance was achieved when the batch size was set to be equal to the length of a session. Moreover, one training session was used for each update cycle as it is the only way that simultaneously acquired neural recordings can be associated with corresponding cursor kinematics. The same FENet parameters are applied to all the neural electrodes. The output of the FENet is a feature matrix with the dimension of B×(N×M), where M is the number of the generated neural features per electrode. This feature generation process is the first stage of the two-stage optimization process. To reduce the dimension of the FENet output per channel to avoid overfitting of the consequent decoder, the electrode specific partial least-squares regressor (PLSR) 374 was applied to the FENet generated features of each neural electrode to reduce the M features to K, in which K≤M The output of the feature extraction network, which was applied on a single session at the current iteration, was used to train an analytical linear decoder, which learns to map the extracted neural features to the movement kinematics of the computer cursor for the current single session analytically by the below formulas:

P=Uβ+ϵ Equation 2

β=(U^{T}U)⁻¹U^{T}P Equation 3

where P is the B×2 kinematics matrix, U is the B×K extracted neural feature matrix, p is the linear decoder coefficients, and c is the regression error. Since predicting the velocity of the cursor movements in a BMI system is more stable and smoother than predicting the cursor position, the cursor velocity was first predicted by using the decoder. Then, to find the position of the cursor movements, the predicted velocity patterns of the cursor in X and Y directions was integrated. After the linear decoder predictions were output, the trained linear decoder parameters were frozen and backpropagation was performed to only update the weights of the feature extraction network. The whole process was repeated to train the example feature extraction network and linear decoder parameters per system update, which happened per session.

For the symmetric replication of the feature engineering modules of the example feature extraction network, the example FENet was designed to have a hierarchical and symmetric architecture similar to the db20 wavelet transform. Since the FENet architecture is inspired by the wavelet transform architecture, the FENet convolutional filters were initialized with db20 mother wavelet filters to guarantee the convergence of the FENet by a more accurate initial condition at the beginning of training. Seven back-to-back feature engineering modules in the FENet architecture were used as shown in FIG. 3B. The length of the convolutional filter for each feature engineering module was set to 40, similar to the length of db20 filters. The convolutional filters kernel sizes and the strides of the filters were set to 1 and 2 for all the convolutional filters, respectively. To compensate for the left and the right edge effect of the inputs to the convolutional filters during the convolution operation, 39 zeros were padded to both sides of the inputs at the first block of the feature engineering modules, which is one less than the filter length to make sure the first convolution only covers the first sample of each input. To tune the network parameters and to train the network, the open-loop neural data recorded from 11 sessions of the first year implants were implanted into the participant JJ was used.

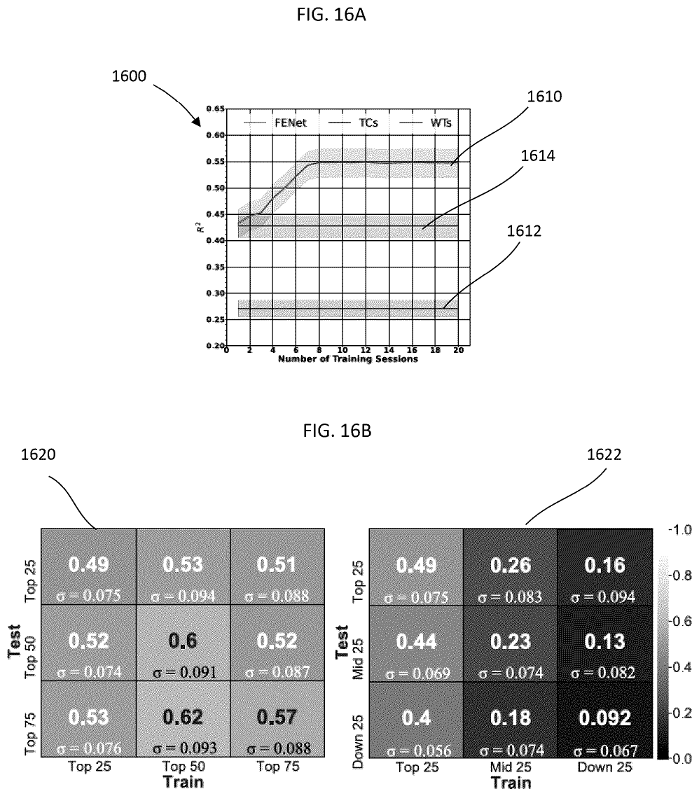

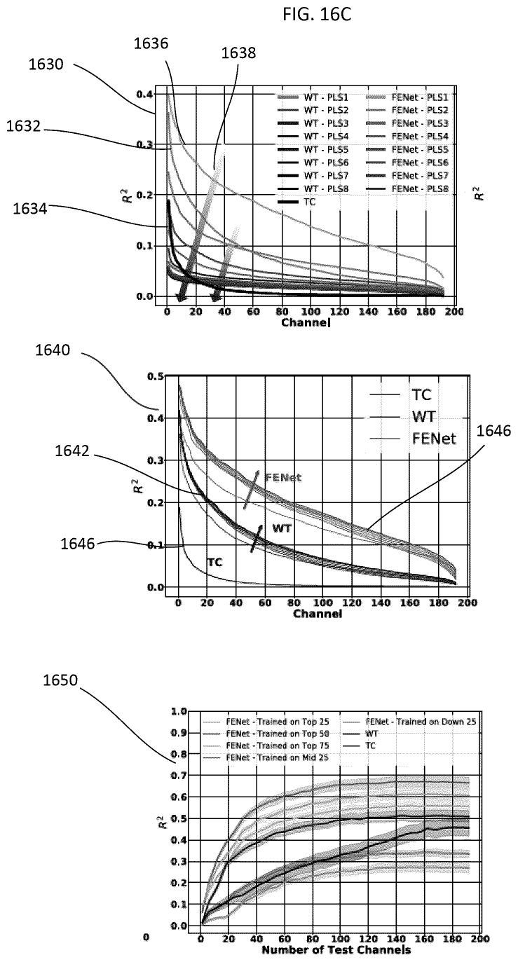

FIG. 16A shows a graph 1600 that plots the coefficient of determination against the number of training sessions, showing the amount of training data needed to train the example FENet, and the known TCs and WTs methods. A plot 1610 shows the coefficient of determination of the example FENet, a plot 1612 shows the coefficient of determination of TCs, and a plot 1614 shows the coefficient of determination for WTs. The number of FENet training sessions was changed from 1 to 10 for each left-out test session. These training sessions were picked from all the available training sessions randomly and this process was repeated 10 times for each left-out test session to report the cross-validated performance. The graph 1600 shows that the performance of the linear decoder saturates by using about 7 sessions for training. Shaded regions show the 95% confidence intervals. To train FENet, cross-validation was performed by dividing the training sessions into train and validation sessions, holding three of the sessions out for validation, while training the network on the remaining eight sessions. For training the linear decoder after FENet generated the features per session, the 10-fold cross-validation on each session was applied. To avoid overfitting, early stopping was used to stop the training when the validation loss on the left-out validation sessions started to increase. Dropout was also employed, which has been shown to reduce overfitting in neural networks. To control the range of the values of the network weights, weight decay regularization was applied on all the weights of the network and batch normalization was applied on the output features as other regularization techniques for the stability of training. The mean square error (MSE) between the predicted and the ground-truth movement kinematics was normalized by using the Adam optimizer to update the learnable parameters of the example FENet. The learning rate α starts at α=0.1, which is divided by 2 every ten epochs using a linear scheduler. The value for the drop-out was set to 0.2 for all the layers. To avoid overfitting of the linear decoder, the batch size was set to be equal to the length of the input session, which is around 20 (sd.+/−3) times greater than the dimensionality of the FENet generated features but can differ from session to session. Early stopping was applied as another regularization technique, which avoids overfitting by stopping the training process if validation loss does not decrease after epochs.

Parameter sweeps were conducted using Bayesian optimization on the FENet model to assess the importance and impact of each hyperparameter in the architecture of the FENet model. The results indicate a correlation between the coefficient of determination, R² values and the parameter values, as explained above in reference to FIGS. 10A and 10B. Notably, the strides of the initial layers emerged as the most influential parameters, with smaller strides yielding higher performance. This is because smaller strides allow the convolutional kernels to cover a greater variety of patterns in the input. Conversely, larger strides limit the coverage between consecutive kernel movements, resulting in the filters learning fewer patterns. Additionally, the kernel size becomes more crucial in later layers compared to the initial layers. This suggests that the inputs to later layers summarize information from multiple samples in the preceding layers. Consequently, the network becomes more sensitive to kernel size when combining richer features with different kernel sizes, as these layers combine samples providing less abstract information than deeper layers.

The training architecture assumes that the neural activity is informative of movement kinematics. Since the example feature extraction network, FENet, was trained on single electrodes, to remove the noisy and non-informative electrodes during training, the example feature extraction network was trained on the top 25, 50, and 75 electrodes with the highest cross-validated values after sorting the neural electrodes according to the values of the TCs with respect to the cursor movement kinematics. FIG. 16B shows a first table 1620 showing training and testing for the top 25, top 50, or top 75 electrodes and a second table 1622 showing training and test for the top 25, mid 50, or down 75 electrodes. The tables 1620 and 1622 show that features generated by an example feature extraction network optimized on the 50 top electrodes have higher averaged performance and is more generalizable to the electrodes that were excluded from training compared to the other feature extraction techniques and parameters. According to the analysis, the top 50 electrodes out of 192 recorded electrodes per session were providing the highest averaged performance on the validation data and therefore were used during training the example feature extraction network FENet. Pre-selecting electrodes based on performance of the threshold crossings method could bias results to favor TCs in comparisons. Despite this, the results from the TCs were consistently outperformed by both the example FENet and the Wavelets, as shown in the closed- and open-loop results. To ensure that there was no feature bias favoring well-tuned electrodes as compared to the other electrodes, the top 75 electrodes of each session were divided into three equal groups of the top 25, middle 25 (mid 25), and bottom 25 (down 25) electrodes based on the sorted cross-validated coefficient of determination values. In each experiment, the example feature extraction network FENet was trained on the top 25, mid 25, or down 25 electrodes of the training sessions separately. Then, the linear decoder was trained and tested when it operated on FENet features extracted from the top 25, mid 25, or down 25 electrodes of the test session. The table 1622 in FIG. 6B shows the cross-validated averaged coefficient of determination, R² of each experiment. The linear decoder operating on the example feature extraction network, FENet. trained on the top 25 electrodes of the training sessions achieved higher averaged R² when the linear decoder was tested on the top 25, the mid 25, or the down 25 electrodes. Therefore, using informative features to train the FENet is an integral aspect of the training process.

During the inference, the trained example feature extraction network FENet was frozen. To be consistent with the training, electrode-specific partial least-squares regression (PLSR) was applied to the generated features of each neural electrode to reduce the M features to K features, in which K≤M. M=8 and K=2 in the experiments according to the analysis on the number of partial least square coefficients (PLSs) needed for regression.

To pick the optimum number of features per electrode for FENet and WTs, the 10-fold cross-validated coefficient of determination of single-electrode TCs, FENet, and WTs were compared using different number of output features. Results are shown separately for each PLS-based latent dimension after sorting the electrodes by maximum per-session coefficient of determination and then averaging the coefficient of determination across the sessions. Electrodes were sorted based on the coefficient of determination value between the ground-truth and the linearly regressed movement kinematics using each single electrode. FIG. 16C shows a graph 1630 that plots the average coefficient of determination for the output channels for WT, TC, and the example FENet in plots 1632, 1634, and 1636 for performance of single PLSR generated features. The direction of arrows 1638 in the graph 1630 show the increase in performance as more PLS features are included per electrode for decoding.

The performance of a linear decoder operating on cumulative PLS features of single electrode, starting from the best PLS feature (e.g., PLS feature 1, 1&2, 1&2&3, etc.) was also examined. A graph 1640 shows the averaged coefficient of determination values plotted against number of channels that shows the cumulative performance of PLSR generated features for WT, TC and the example FENet in plots 1642, 1644, and 1646 respectively. The graph 1640 shows that top two WTs and FENet PLS features are enough for the linear decoder to reach approximately maximum performance. Thus, the features are limited to the top two PLS features for the population-based reconstructions of movement kinematics. Limiting the number of features prevents an explosion of predictive features that can result in overfitting and poor generalization.

A graph 1650 shows Neuron dropping curves. To generate the neuron dropping curves, a group of electrodes was randomly picked from all the available 192 electrodes. The performance of the decoder was tested on the selected electrodes. This process was repeated 100 times for each group size. The group size can vary from 1 (i.e., a single electrode) to 192 (all electrodes). Neuron dropping curves were generated on the neural data of participant JJ on the sessions recorded in 2019. The graph 1650 shows the performance of FENets that are trained on top 25, top 50, top 75, mid 25, and down 25 electrodes as well as the performance of the WTs and TCs. FENet trained on the top 50 electrodes shows superior performance and generalizability compared to the other techniques.

The Partial Least Squares Regression (PLSR) maps the input features to a lower-dimensional space by defining an analytic linear transformation between its inputs and its lower dimensional outputs, which maximizes the covariance between the neural data and the kinematics. Then an analytical linear decoder was trained based on the top two PLS-generated neural features to minimize overfitting that can occur when too many predictor variables are used relative to the amount of the training data.

In order to evaluate the impact of PLSR on the performance of the linear decoder operating on the example feature extraction network FENet, a rigorous analysis utilizing data from all 54 sessions of participant JJ was conducted as shown in the graphs 730 and 740 in FIG. 7B. The evaluation involved a comparison of the performance of the example feature extraction network with and without the application of PLSR, specifically applied to the top 40 electrodes within these sessions. The selection of these top 40 electrodes was motivated by the goal of mitigating potential overfitting issues that may arise in the linear decoder, particularly in scenarios where PLSR is not employed. The results depicted in the graphs 730 and 740 demonstrate that the example FENet exhibits the ability to effectively capture informative features from the vast neural data, irrespective of the presence or absence of PLSR. Additionally, the application of PLSR plays a vital role in reducing the dimensionality of the extracted features. This dimensionality reduction step is crucial as it helps prevent overfitting of the decoders, particularly when working with limited neural data obtained from human participants within each session. These findings highlight the robustness and efficacy of the example feature extraction network (FENet) as a feature extraction technique in neural decoding tasks. Furthermore, they underscore the importance of employing dimensionality reduction methods such as PLSR, which can enhance the performance and generalizability of the linear decoder by mitigating the risk of overfitting when working with limited neural data.

To assess the significance of each extracted feature by the example feature extraction network for every electrode, the Shapley value was employed as a measure of importance. The Shapley value allows determination of the contribution of each input feature in the decoding process when utilizing a linear decoder. The computation of the Shapley value involves comparing the output of the decoder with and without the inclusion of a specific feature. The discrepancy between these two cases reflects the contribution of the feature to the decoding process. This calculation is repeated for all possible combinations of features per electrode, and the Shapley value for a given feature is determined by averaging these contributions across all possible combinations, taking into account the number of combinations that include the feature. In this manner, the incremental contribution of each feature to the output of the decode can be evaluated while considering the interactions between features. Features with higher Shapley values are deemed more important since they make a greater contribution to the output variable compared to other features. As explained above the graphs in FIGS. 10A-10C present the relative Shapley values of the eight FENet-extracted features. These values represent the average contribution of each feature to the decoding process, calculated and averaged across all electrodes and sessions, using offline data recorded from the participant JJ during the center-out task. The graphs in FIGS. 10A-10C illustrate that the features extracted at the initial stage play a more crucial role in predicting outcomes through the linear decoder.

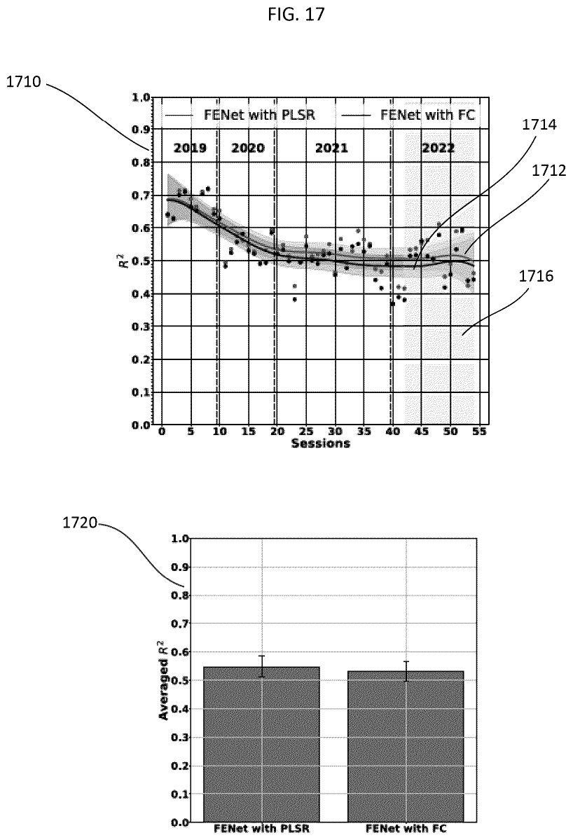

As an alternative to the PLSR for dimensionality reduction, to combine the dimensionality reduction technique with the feature extraction process, the PLSR may be replaced with a single fully connected layer as the last layer of the FENet, which maps the eight example generated features to one feature per electrode. FIG. 17 shows a graph 1710 plotting the coefficient of determination of the example FENet when the dimensionality of the FENet convolutional filters outputs is reduced by using the PLSR (plot 1712) or the added fully connected layer as the last layer of the example FENet (plot 1714) for dimensionality reduction. A shaded region 1716 shows the closed-loop sessions. The band in each time series shows the range of its 95% confidence interval of a LOESS fit. A graph 1720 shows the averaged coefficient of determination for the example FENet with the PLSR and with the added fully connected layer. According to the graphs 1710 and 1720, the performance of the decoder stays almost the same independent from these two dimensionality reduction techniques. Combining the feature extraction and the dimensionality reduction processes makes the use of the example feature extraction network architecture easier. However, there is less control on the number of extracted features per electrode compared to using the PLSR for dimensionality reduction.

To evaluate the performance of different feature extraction techniques, the features were passed to different types of decoders, including a Linear Decoder, a Support Vector Regression decoder, a Long-Short Term Recurrent Neural Network (LSTM) decoder, a Recalibrated Feedback Intention-Trained Kalman filter (ReFIT-KF) decoder, and a Preferential Subspace Identification (PSID) decoder as explained above.

The Linear Decoder used a standard linear regression model where kinematics (ŷ) may be predicted from the extracted neural features (u) by using:

ŷ=b+Σᵢ₌₁^{N}Wᵢuᵢ Equation 4

The weights and the bias term are found through a least squares error optimization to minimize mean squared error between predictions of the models and ground-truth kinematics during training. The parameters are then used to predict new kinematics given extracted neural features.

Support vector regression (SVR) is the continuous form of support vector machines where the generalized error is minimized, given by the function:

ŷ=Σᵢ₌₁^{N}(αᵢ*−α₁)k(uᵢ,u)+b Equation 5

Where αᵢ* and αᵢ are Lagrange multipliers and k is a kernel function, where the radial basis function kernel is used. The Lagrange multipliers are found by minimizing a regularized risk function:

$${R}_{reg} [ f ] = \frac{1}{2} {‖ w ‖}^{2} + C {\sum }_{i = 1}^{l} {L}_{ε} ( y ) \qquad Equation 6$$

where ∥w∥² represents the model complexity, C is a constant that determines the ε trade-off between the insensitive loss function L_{ε}(y). For SVR, an RBF kernel was employed with C set to 1.

It is well-known that simple RNN units cannot remember long term dependencies in sequential data because of the vanishing gradients problem. Another version of RNNs that is widely used in the literature are RNNs with Long-Short Term Memory (LSTM) units. By denoting º as the Hadamard product, the LSTM is defined as:

$$\{ \begin{matrix}{f}_{k} = σ ( {W}_{fu} {u}_{k} + {W}_{fr} {r}_{k - 1} + {b}_{f} ) \\ {i}_{k} = σ ( {W}_{i u} {u}_{k} + {W}_{ir} {r}_{k - 1} + {b}_{i} ) \\ {o}_{k} = σ ( {W}_{o u} {u}_{k} + {W}_{or} {r}_{k - 1} + {b}_{i} ) \\ {c}_{u} = tanh ( {W}_{c u} {u}_{k} + {W}_{cr} {r}_{k - 1} + {b}_{c} ) \\ {c}_{k} = {f}_{k} ∘ {c}_{k - 1} + {i}_{k} ∘ {c}_{k - 1} \\ {r}_{k} = {o}_{k} ∘ tanh ( {c}_{k} ) \\ {\hat{y}}_{k} = {W}_{y r} {r}_{k} + {b}_{y}\end{matrix} \qquad Equation 7$$

γₖ is the hidden state as in a simple RNN, cᵤ is the output from the cell update activation function, cₖ is the LSTM cell's internal state, fₖ, iₖ, and oₖ are the output matrices from the respective forget, input, and output activation functions, which act as the gates of the LSTM, and represent the weights and biases, and a is the sigmoid function. Following parameter sweeps, the determined settings were 1 layer, 50 recurrent nodes, and the history of LSTM was 10.

The Kalman Filter decoder combines the idea that kinematics are a function of neural firings as well as the idea that neural activity is a function of movements, or the kinematics. This can be represented by two equations:

$$\{ \begin{matrix}{\hat{y}}_{k + 1} = {A}_{k} {\hat{y}}_{k} + {w}_{k} \\ {u}_{k} = {H}_{k} {\hat{y}}_{k} + {q}_{k}\end{matrix} \qquad Equation 8$$

These represent how the system evolves over time as well as how neural activity is generated by system's behavior. The matrices A, H, Q, and W can be found through a training process (where q˜N(0, Q) and w˜N (0, W). Using properties of the conditional probabilities of kinematics and neural data, a closed form solution is obtained for maximizing the joint probability p (Y_{M}, U_{M}). Using the physical properties of the problem, a matrix A is obtained to be of the form:

$$A = [ \begin{matrix}1 & 0 & d t & 0 \\ 0 & 1 & 0 & d t \\ 0 & 0 & a & b \\ 0 & 0 & c & d\end{matrix} ] \qquad Equation 9$$

Where Aᵥ is defined as:

$${A}_{v} = [ \begin{matrix}a & b \\ c & d\end{matrix} ] = {V}_{2} {{V}_{1}^{T} ( {V}_{1} {V}_{1}^{T} )}^{- 1} \qquad Equation 10$$

where V₁ consists of the velocity kinematics points except for the last time step, V₂ consists of the velocity kinematics points except for the first time step, and dt is the time step size used. In this case, the time step was 30 ms for participants JJ and NS, and 50 ms for participant EGS). Furthermore, W is a zero matrix with the matrix

$${W}_{u} = \frac{1}{N - 1} ( {V}_{2} - A {V}_{1} ) {( {V}_{2} - A {V}_{1} )}^{T}$$

in the bottom corner. H and Q are given by:

$$\{ \begin{matrix}H = {U}^{T} {Y ( {YY}^{T} )}^{- 1} \\ Q = \frac{1}{N} ( U - HY ) {( U - HY )}^{- 1}\end{matrix} \qquad Equation 11$$

Then, the updated equations can be used.

$$\{ \begin{matrix}{\hat{y}}_{k}^{-} = A {\hat{y}}_{k - 1} \\ {P}_{k}^{-} = A {P}_{k - 1} {A}^{T} + W \\ {\hat{y}}_{k} = {\hat{y}}_{k}^{-} + {K}_{k} ( {u}_{k} - H {\hat{y}}_{k}^{-} ) \\ {P}_{k} = ( 1 - {K}_{k} H ) {P}_{k}^{-}\end{matrix} \qquad Equation 12$$

where P is the covariance matrix of the kinematics, Kₖ, the Kalman filter gain is given by:

Kₖ=Pₖ⁻H^{T}(HPₖ⁻H^{T}+Q)⁻¹ Equation 13

The PSID decoder models the state of the brain as a high-dimensional latent variable influencing neural activity and behavior. PSID is an algorithm built upon the Kalman Filter equations and utilizes a dynamic linear state space model to describe the association between the latent state and the recorded neural activity (uₖ) and behavior (yₖ). The model consists of a latent state xₖ∈ [symbol] , which includes behaviorally relevant (xₖ⁽¹⁾∈ [symbol] ) and irrelevant (xₖ⁽²⁾∈ [symbol] ) components as below:

$$\{ \begin{matrix}{x}_{k + 1} = A {x}_{k} + {w}_{k} \\ {u}_{k} = {C}_{y} {x}_{k} + {v}_{k} \\ {y}_{k} = {C}_{z} {x}_{k} + {ε}_{k}\end{matrix} \qquad Equation 14$$

$${x}_{k} = [ \begin{matrix}{x}_{k}^{( 1 )} \\ {x}_{k}^{( 2 )}\end{matrix} ]$$

PSID employs a two-stage identification approach. In the first stage, PSID directly learns the behaviorally relevant component (xₖ⁽¹⁾) from training data without simultaneously learning the irrelevant component (xₖ⁽²⁾), which is optional in the second stage. This prioritization enables the PSID model to learn behaviorally relevant neural dynamics using low-dimensional states (only xₖ⁽¹⁾). Similar to a Kalman filter, the PSID model formulation includes noise terms (∈ₖ, wₖ, and vₖ) representing behavior dynamics not present in the recorded neural activity. The parameters of the model (A, C_{y}, C_{z}, and noise statistics) are learned by the PSID using training samples of neural activity and behavior. After the parameter sweep, the latent space dimension was adjusted to 10.

The open-loop evaluation measure was determined as follows. The cross-validated coefficient of determination, R², is reported as a measure of the strength of the linear association between the predicted and the ground-truth kinematics, respectively. The Rₓ⁽²⁾ and R_{y}⁽²⁾ were computed independently in the X (horizontal) and Y (vertical) dimensions using the definition of the coefficient of determination:

$${R}^{2} = {( \frac{{\sum }_{i} ( {y}_{i} - \overline{y} ) ( {\hat{y}}_{i} - \overline{\hat{y}} )}{\sqrt{{\sum }_{i} {( {y}_{i} - \overline{y} )}^{2}} \sqrt{{\sum }_{i} {( {\hat{y}}_{i} - \overline{\hat{y}} )}^{2}}} )}^{2} \qquad Equation 15$$

where yᵢ and ŷᵢ are the i^{th} ground-truth and prediction, respectively. R² is a real number varies from 0 to 1. The larger the cross-validated coefficient of determination is, the better the performance. Results are qualitatively the same when analyzing each dimension separately. Then, the combined R² value was calculated for both X and Y directions to be the norm of the [Rₓ⁽²⁾, R_{y}⁽²⁾] vector as below:

$${R}^{2} = \frac{1}{\sqrt{2}} \sqrt{{( {R}_{x}^{2} )}^{2} + {( {R}_{y}^{2} )}^{2}} \qquad Equation 16$$

The maximum for R² occurs when the predictions and the ground-truth are completely matched, in which Rₓ⁽²⁾ and R_{y}⁽²⁾ are both equal to 1.

To assess the performance on the finger-grid task, the framework of representational similarity analysis (RSA) and representational dynamics analysis (RDA) was employed. RSA quantifies the neural representational structure by measuring the pairwise distances between the neural activity patterns associated with each finger. These distances are used to construct the representational dissimilarity matrix (RDM), which provides a concise summary of the representational structure. Notably, these distances are independent of the original feature types, such as electrode or voxel measurements, enabling comparison of finger organizations across subjects and different recording modalities. Additionally, representational dynamics analysis (RDA) was utilized to explore the temporal evolution of the representational structure. This involved modeling the representational structure of finger movements at each timepoint as a non-negative linear combination of potentially predictive models.

To compare the improvement of the predictability of each single electrode using different feature extraction techniques, three distinct linear decoders were trained, one per each for the example feature extraction network FENet, the TC features, and the WT features that were extracted from each single electrode. Then, the movement kinematics for each of these three decoders were predicted corresponding to three single-electrode features. Finally, the cross-validated values of the predictions for each single neural electrode were compared. This process was repeated for all the other electrodes of 11 sample recording sessions for participant JJ. The graphs in FIGS. 8A-8C show the value of linear decoder operating on FENet, WTs, and TCs as the feature extraction technique with respect to each other, in a series of pair-wise comparisons. The cluster of dots 814 represent the electrodes that have had low values in both feature extraction techniques, whereas the cluster of dots 812 represent the electrodes with the high values in at least one of the reported feature extraction techniques. FENet improved single-electrode values compared to the TCs (Binomial test, p=0) and the WTs (Binomial test, p=4e-8).

To compare the preferred tuning direction of the FENet features per channel, three distinct linear decoders were trained, one for each feature extraction technique (FENet, TCs, WTs) per channel. Then, the phase and the magnitude difference between the corresponding tuning vectors for each pair of feature extraction techniques were calculated as shown in FIG. 8B. Although the feature extraction techniques are inherently different, activity of a similar electrode maintains its preferred direction independent from a specific feature extraction technique.

Several metrics were used to evaluate the closed-loop decoding performance: success rate as the number of correct trials completed within a fixed amount of time; time required for the cursor to reach the target; the path efficiency as measured by the ratio of path-length to straight-line length; the instantaneous angular error that captures the angle between a vector pointing towards the target and the instantaneous velocity of the cursor; accuracy (how well the cursor tracks participant intentions); and blinded queries to research participants to evaluate responsiveness (how quickly the cursor responds to participant intentions). In addition, for the grid-task, the bit rate is included in the findings. The calculation of the bit rate is outlined below:

$$B = \frac{{log}_{2} ( N ) \times {S}_{c}}{r} \qquad Equation 17$$

where N is the number of total targets on the screen, Sc is the number of completed trials, and t is the time elapsed in seconds. The computational overhead was evaluated by tracking how much time is required to compute each prediction's update. With this array of metrics, a more complete picture of the performance and computational consequences of the design choices, and their impact on the participants' user experience and preference was built. This evaluation shows the example feature extraction network resulted in improved closed-loop performance.

The ability to test the example feature extraction network using neural recordings during development and operation with human participants during test and validation is critical to validating the success of the example feature extraction network. The testing of the feature extraction techniques included both data-driven measurements of performance as well as quantitative and subjective feedback provided by human research participants during double-blind testing. The double-blind testing was used to capture quantifiable and subjective performance metrics of the algorithms being tested for each of the feature types (TCs and FENet). In each session, these two feature extraction techniques (hereafter techniques A and B) were selected for evaluation. One batch consisted of an open-loop training run with 64 trials to parameterize A and B, a single closed-loop re-training run with 64 trials to re-train A and B decoders, and two closed-loop runs per algorithm each with 96 trials (four total closed-loop runs, with A and B shuffled). Each run lasted approximately 3-5 minutes, for a total of 15-25 minutes per batch. Two batches were performed in each session with at least a ten-minute break between and alternating the starting algorithm between sessions. The participant and researchers had been told which algorithm was being used (“A” or “B”) but not what A or B were. After each batch, the participant was queried to capture subjective experience and preference in each session.

In order to determine the computational complexity of various architectures for the example feature extraction network, the total count of multiplicative and additive operations performed for the feature extraction within the network were quantified. It was assumed that Sᵢ, kᵢ, and sᵢ are input size, kernel size, and stride of the i^{th} feature engineering module of the example feature extraction network, respectively. The size of the input for the i^{th} feature engineering module can be calculated as below:

$${S}_{i} = ⌊ \frac{{S}_{i - 1} + max ( {k}_{i} - {s}_{i} , 0 ) + ( {k}_{i} - 1 ) - {k}_{i}}{{s}_{i}} ⌋ \qquad Equation 18$$

where max (kᵢ−sᵢ, 0) and (kᵢ−1) represent the left and right paddings, respectively. Then, the cost for all the layers may be calculated as below:

Cost=Σᵢ₌₀ⁿ⁻¹2kᵢSᵢ Equation 19

given that n represents the quantity of feature engineering modules within the FENet, it is necessary to consider the dual cost incurred by both the upper and lower branches of these modules. As such, the computational cost is effectively doubled to encompass the collective operations of these components.

The programmatical framework to train and operate neural networks in the experiments was PyTorch, a deep-learning API for Python. Pytorch was configured to use CUDA, a parallel computing platform and programming model developed by NVIDIA, which can accelerate many of the computations involved in training neural networks with commercially available graphics processing units (GPU). For offline training and evaluation of the example feature extraction network, FENet, a single Tesla V100 GPU was used and for the closed-loop runs, a single NVIDIA GeForce RTX 3080 GPU was used.

Offline the example FENet-based features outperform these outputs of the two known feature extraction methods (TC and WT), decreasing mean square error by 50 and 47 percent, respectively as shown in the graphs 550 and 560 in FIG. 5C. The example feature extraction network, FENet is robust, demonstrating improved performance with significantly reduced daily variability. The example feature extraction network is generalizable, providing improved performance when applied to signals recorded from individuals and brain regions not used to train the algorithm. The features from the example feature extraction network enable a 2 to 3 times improvement in online performance metrics such as the bitrate of communication and angular error of cursor trajectories as shown in the graphs in FIG. 4B. Critically, the patient demonstrating these improvements had implants for 3.5 years with neural signals degrading to the point that the patient desired a reimplantation surgery. The example feature extraction network enabled sufficient improvement in control that the patient no longer required a new set of implants. The results demonstrate that the trained feature extraction network, constrained by domain-specific knowledge, can be used for novel datasets, without modification, to significantly improve the performance, generalization, and efficiency of training. Thus, the example novel algorithmic solution incorporated in the example feature extraction network allows extend the lifetime of implanted electrode arrays and to generally improve the performance of BMI systems using a simple drop-in solution of a system coupled to existing implants.

In a similar vein to conventional feature extraction methods that apply uniform operations to individual electrodes, the example feature extraction network employs a consistent feature extraction process across all electrodes. Future BMI systems may not universally support the capture of raw, high-sample rate broadband data (e.g., Neuralink). In such cases, the example feature extraction network approach can be seamlessly utilized without the reliance on this type of data. Additionally, training a neural network using data from all electrodes presents a more challenging learning problem, as the variations across electrodes may change over time. Consequently, this limitation constrains the potential benefits of hyper-specific solutions tailored to individual electrodes. Therefore, the example feature extraction network is designed to be agnostic to the specific number and configuration of electrodes within different BMI systems, making it readily adoptable by users, particularly those who prefer to avoid setting up their own training protocols.

Interestingly, across all testing conditions, FENet improved results when analysis was done at fine temporal scale. However, in some cases, the benefits of FENet were reduced as smoothing was applied to the data. Thus, FENet seems to significantly reduce high-frequency within-trial variability, however, may have less impact on reducing trial-to-trial variability as shown in the graph 1550 in FIG. 15, depending on experimental setup. Reducing the high-frequency variability is critical for real-time BMIs, situations in which the behavior unfolds over very rapid timescales, when looking for precise estimates of timing, or when attempting to infer network dynamics. Trial-to-trial variability can be captured by how similar the neural response is when repeating the same experimental trial. The trial-to-trial variability is an accurate report of how behavioral factors have changed the activity measured at the single electrode level. Reducing the trial-to-trial variability is also important, however, the underlying reasons for trial-to-trial variability are less clear. For example, measured differences may be accurate reflections of the underlying state of the network that e.g., reflect task irrelevant features of the patient's behavioral state. To the degree that trial-to-trial differences are driven by behavioral factors, no feature extraction algorithm measuring the local activity of neurons can reduce this variability from the perspective of a feature extraction technique recording activity from an electrode.

To overcome the constraints imposed by the limited opportunities for closed-loop testing, an offline analysis was conducted to compare the performance of the example FENet with multiple other feature extraction techniques. Although direct comparison in closed-loop testing is ideal, it is challenging to achieve frequently. To expand the scope of comparison across different time periods and feature extraction techniques, the capability of the example FENet to reconstruct movement kinematics using previously recorded neural data from implanted electrode arrays was evaluated.

The FENet was designed to maintain a small computational footprint in comparison to hypothetical ultradeep RNN feature extraction techniques and other convolutional network designs. This was achieved by extracting features from single electrodes using the same trained parameters for all electrodes. The example architecture was constrained to an algorithm with complexity that allows for computation within 5 milliseconds in closed-loop BMI. The example FENet, based on the wavelet with db20 mother wavelet architecture described above consists of only 560 learnable parameters. This significantly reduces i size compared to more complex deep-network alternatives. Additionally, swapping hyperparameters of the example FENet demonstrates that comparable benefits and performance may be achieved even with a smaller architecture.

Traditionally, BMI systems can trade-off speed and accuracy depending on the design preferences. The ability of the example feature extraction network to improve on both sets of metrics in parallel represents a significant advance in BMI design. Importantly, these advantages come with little or no cost in either computational or experimental performance. The example feature extraction network preserves the representational structure of sorted neural populations and therefore is applicable to any subsequent decoding scheme. Moreover, the example FENet improved the ability of a test participant to use brain signals to control a computer cursor, increasing the bitrate nearly threefold in the closed-loop control. The incorporation of the example FENet can extend the functional lifetime of implanted electrodes, mitigating the need for revision surgeries and thus improving commercial viability. The improved performance specifically pertains to the feature extraction component, where the patient serves as their own control.

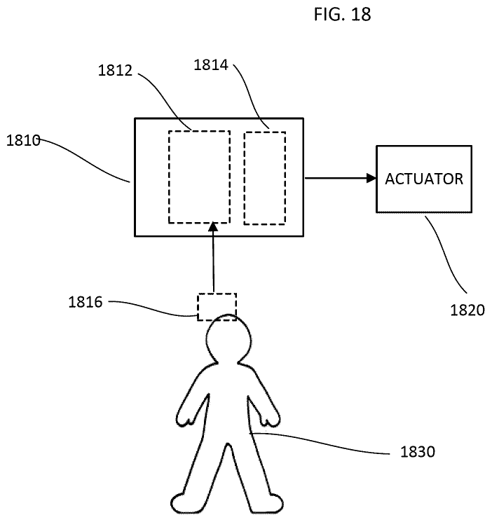

The example feature extraction network may be trained to receive broadband data from a single neural sensor such as an electrode and extract the most informative features automatically. This training procedure can be replicated for all recording sensors to assess the current neural population state, regardless of the application and without reliance on the decoder. Thus, the decoder can be substituted with a classifier or regressor to suit the specific application requirements. Thus, the trained feature extraction network and trained decoder architecture may be used in applications other than the cursor based tasks. FIG. 18 shows a block diagram of a trained brain computer interface 1810 controlling an actuator 1820. In this example, the brain machine interface 1810 includes a feature extraction network 1812 having a series of feature engineering modules and a decoder 1814 that are trained by the process described above. A set of neural implants 1816 provides neural inputs from a subject 1830 to the brain machine interface 1810. The electrodes of the neural implants 1816 are neural sensors. The neural signals are input into the feature extraction network 1812 of the brain machine interface 1810. The feature engineering modules of the feature extraction network 1812 output a set of features. The kinematics data is output from the decoder 1814 based on the features extracted from the neural network 1812. The kinematics data from the decoder 1814 is output to an output interface. The output interface is coupled to the actuator 1820, thus allowing the subject 1830 to control the actuator 1820.

Another application may be brain state estimation, such as identifying brain states in Alzheimer's disease or migraines, as well as in classification tasks like seizure detection. Another application may be in classification of brain disorders. A classification system may incorporate the trained feature extraction network and a decoder to output brain state data. The brain state data may be displayed and or analyzed to determine brain diseases and disorders and classification of such diseases and disorders.

The advantages of the example feature extraction network optimizes the information content of neural features that has the advantages of 1) it easily drops into current decoding pipelines across cortical brain regions, patients, and tasks, demonstrating its ability to serve as a drop-in replacement for other feature extraction techniques; 2) generalization across electrodes, patients, brain areas, and implant duration without parameterization; 3) running real-time on standard computers and ultimately be deployable in low-power application specific integrated circuits (ASICs); and 4) not significantly increase the complexity or amount of training data required for the subsequent decoding algorithm that maps the extracted neural features to the participant's intent. This architecture was structured to maximize the amount of information contained in the extracted neural features, while abstracting away the parametric relationship between the extracted features and the decoded participant behavior.

Additionally, the example FENet improves the signal to noise ratio of extracted neural features over the entire lifetime of the array. FENet demonstrated a minimum improvement of ˜50% in cross-validated coefficient of determination (R²) across multiple patients and through the lifetime of the arrays. The population-level analysis demonstrated that FENet preserves the representational structure and temporal dynamics of sorted neural populations and, thus, provides an accurate measure of brain activity. Taken together, FENet can improve the efficacy of implantable electrode systems while delivering improved performance and ease of use.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In one or more embodiments, computer-executable instructions are executed on a general purpose computer to turn the general purpose computer into a special purpose computer implementing elements of the disclosure. The computer executable instructions may be, for example, binaries, intermediate format instructions such as assembly language, or even source code. Although the subject matter has been described in language specific to structural marketing features and/or methodological acts, it is to be understood that the subject matter defined in the appended claims is not necessarily limited to the described marketing features or acts described above. Rather, the described marketing features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as an un-subscription model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing un-subscription model can be composed of various characteristics such as, for example, on-demand self-service, broad network access, resource pooling, rapid elasticity, measured service, and so forth. A cloud-computing un-subscription model can also expose various service un-subscription models, such as, for example, Software as a Service (“SaaS”), a web service, Platform as a Service (“PaaS”), and Infrastructure as a Service (“IaaS”). A cloud-computing un-subscription model can also be deployed using different deployment un-subscription models such as private cloud, community cloud, public cloud, hybrid cloud, and so forth. In this description and in the claims, a “cloud-computing environment” is an environment in which cloud computing is employed.

In one example, a computing device may be configured to perform one or more of the processes described above. the computing device can comprise a processor, a memory, a storage device, an I/O interface, and a communication interface, which may be communicatively coupled by way of a communication infrastructure. In certain embodiments, the computing device can include fewer or more components than those described above.

In one or more embodiments, the processor includes hardware for executing instructions, such as those making up a computer program. As an example and not by way of limitation, to execute instructions for digitizing real-world objects, the processor may retrieve (or fetch) the instructions from an internal register, an internal cache, the memory, or the storage device and decode and execute them. The memory may be a volatile or non-volatile memory used for storing data, metadata, and programs for execution by the processor(s). The storage device includes storage, such as a hard disk, flash disk drive, or other digital storage device, for storing data or instructions related to object digitizing processes (e.g., digital scans, digital models).

The I/O interface allows a user to provide input to, receive output from, and otherwise transfer data to and receive data from computing device. The I/O interface may include a mouse, a keypad or a keyboard, a touch screen, a camera, an optical scanner, network interface, modem, other known I/O devices or a combination of such I/O interfaces. The I/O interface may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface is configured to provide graphical data to a display for presentation to a user. The graphical data may be representative of one or more graphical user interfaces and/or any other graphical content as may serve a particular implementation.

The communication interface can include hardware, software, or both. In any event, the communication interface can provide one or more interfaces for communication (such as, for example, packet-based communication) between the computing device and one or more other computing devices or networks. As an example and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI.

Additionally, the communication interface may facilitate communications with various types of wired or wireless networks. The communication interface may also facilitate communications using various communication protocols. The communication infrastructure may also include hardware, software, or both that couples components of the computing device to each other. For example, the communication interface may use one or more networks and/or protocols to enable a plurality of computing devices connected by a particular infrastructure to communicate with each other to perform one or more aspects of the digitizing processes described herein. To illustrate, the image compression process can allow a plurality of devices (e.g., server devices for performing image processing tasks of a large number of images) to exchange information using various communication networks and protocols for exchanging information about a selected workflow and image data for a plurality of images.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above-described embodiments. Rather, the scope of the invention should be defined in accordance with the following claims and their equivalents.

1. A brain interface system comprising:
a set of neural signal sensors sensing neural signals from a brain;
a feature extraction module including a plurality of feature engineering modules each coupled to the set of neural signal sensors, wherein the plurality of feature engineering modules are trained to extract a plurality of features from the sensed neural signals;
a decoder coupled to the feature extraction module, the decoder determining a brain state output from a pattern of the plurality of features.

2. The system of claim 1, wherein the brain state output is a kinematics control, and the system further comprising an output interface providing control signals based on the kinematics output from the decoder.

3. The system of claim 2, wherein the output interface is a display and wherein the control signals manipulate a cursor on a display.

4. The system of claim 2, further comprising a mechanical actuator coupled to the output interface, wherein the control signals manipulate the mechanical actuator.

5. The system of claim 1, wherein the set of neural signal sensors is one of a set of implantable electrodes or wearable electrodes.

6. The system of claim 1, wherein the brain state output is an indication of a brain disorder.

7. The system of claim 1, wherein each of the feature engineering modules include an upper convolutional filter coupled to the neural signal sensors and an activation function to output a feature from the neural signal sensors.

8. The system of claim 7, wherein each of the feature engineering modules include a lower convolutional filter coupled to the neural signal sensors, wherein the lower convolutional filter outputs an abstract signal to a subsequent feature engineering module, and wherein the lower convolutional filter of a last feature engineering module outputs a final feature.

9. The system of claim 8, wherein each of the plurality of feature engineering modules use identical parameters for all neural signal sensors used in a training data set for training the feature engineering modules.

10. The system of claim 7, wherein each of the plurality of feature engineering modules include an adaptive average pooling layer coupled to the activation function to summarize a pattern of features into a single feature.

11. The system of claim 7, further comprising either a partial least squares (PLS) regression module coupled to the output of the feature extraction module or a fully-connected layer of nodes, to reduce the plurality of features to a subset of features.

12. The system of claim 7, wherein the training of the feature engineering modules includes adjusting the convolutional filters from back propagation of error between the brain state output of the decoder from a training data set and a desired brain state output.

13. The system of claim 1, wherein the decoder is one of a linear decoder, a Support Vector Regression (SVR) decoder, a Long-Short Term Recurrent Neural Network (LSTM) decoder, a Recalibrated Feedback Intention-Trained Kalman filter (ReFIT-KF) decoder, or a Preferential Subspace Identification (PSID) decoder.

14. The system of claim 1, wherein a batch normalization is applied to the inputs of a training data set for training the feature engineering modules.

15. A method of deriving features from a neural signal for determining brain state signals from a human subject, the method comprising:
receiving a plurality of neural signals from the human subject via a plurality of neural signal sensors; and
determining features from the plurality of neural signals from a feature extraction network having a plurality of feature engineering modules, each trained to extract a feature from the neural signals.

16. The method of claim 15, further comprising decoding the features via a trained decoder to output brain state signals to an output interface.

17. The method of claim 16, wherein the brain state output is a kinematics control, and wherein the output interface provides control signals for a cursor on a display or a mechanical actuator based on the kinematics output from the decoder.

18. The method of claim 15, wherein each of the feature engineering modules include an upper convolutional filter coupled to the neural signal sensors, a lower convolutional filter coupled to the neural signal sensors, and an activation function to output a feature from the neural signal sensors.

19. The method of claim 18, wherein each of the plurality of feature engineering modules use identical parameters for all neural signal sensors used in a training set for training the feature engineering modules.

20. A non-transitory computer-readable medium having machine-readable instructions stored thereon, which when executed by a processor, cause the processor to:
receive a plurality of neural signals from the human subject via a plurality of neural sensors;
determine features from the plurality of neural signals from a feature extraction network having a plurality of feature engineering modules, each trained to extract a feature from the neural signal; and
decode the features via a trained decoder to output brain state signals to an output device.
